(19) United States
(12) Patent Application Publication
(10) Pub. No.: US 2026/0266813 A1
(43) Pub. Date: Sep. 10, 2026

(54) MAGNETIC PARTICLE SPECTROSCOPY

(71) Applicant: Regents of the University of Minnesota, Minneapolis, MN (US)

(72) Inventors: Jian-Ping Wang, Shoreview, MN (US); Kai Wu, Saint Paul, MN (US); Maxim Chacko-Joseph Cheeran, Apple Valley, MN (US); Vinit Kumar Chugh, Minneapolis, MN (US); Venkatramana Divana Krishna Bhat, Maple Grove, MN (US)

(21) Appl. No.: 18/873,654

(22) PCT Filed: Jun. 14, 2023
(86) PCT No.: PCT/US2023/068433

§ 371 Date: Dec. 10, 2024

Related U.S. Application Data
(60) Provisional application No. 63/366,384, filed on Jun. 14, 2022.

(51) Int. Cl. G01N 33/543; G01N 33/569

(52) U.S. Cl. CPC G01N 33/54333; G01N 33/56983; G01N 2333/165; G01N 2469/10

(57) ABSTRACT

An example method includes receiving a sample in a fluid, the fluid comprising a plurality of surface functionalized magnetic nanoparticles (MNPs), the surface functionalized MNPs comprising a probe configured to capture an analyte. Hie method also includes increasing a rate of capture by which the probe is configured to capture the analyte within the fluid. The method also includes, subsequent to increasing the rate of capture, sensing, via volumetric-based magnetic particle spectroscopy (MPS), a magnetic response of the plurality of surface functionalized MNPs, the magnetic response being indicative of whether the sample comprises the analyte.

This application claims the benefit of U.S. Provisional Patent Application Ser. No. 63/366,384, entitled “MAGNETIC PARTICLE SPECTROSCOPY.” and filed on Jun. 14, 2022.

### GOVERNMENT RIGHTS

This invention was made with government support under DE030832 awarded by the National Institutes of Health, and 2020-67021-31956 awarded by the National Institute of Food and Agriculture. The government has certain rights in the invention.

### TECHNICAL FIELD

This disclosure relates to magnetic particle spectroscopy.

### BACKGROUND

Bioassays are procedures for detecting or measuring the concentration or potency of a substance by its effect on living cells or tissues. Immunoassays are procedures for detecting or measuring specific proteins or other substances through their properties as antigens and/or antibodies. In some instances, immunoassays, among other tests, may be performed using magnetic particle spectroscopy (MPS).

### SUMMARY

In general, this disclosure describes example magnetic particle spectroscopy (MPS) devices and techniques for detecting chemicals and biological substances via MPS.

In some examples, a fast MPS technique and device is described. The fast MPS technique and device may be configured to increase a rate of capture by which a probe is configured to capture an analyte which a sample may contain, and to determine a magnetic response of a sample that is indicative of whether the sample comprises an analyte in less than five minutes from the sample being added to a fluid comprising a plurality of magnetic nanoparticles (MNPs).

The fast MPS technique and device includes adding a sample (e.g., a biological sample from a patient) to a fluid comprising a plurality of functionalized MNPs and incubating the fluid and sample for an incubation time period. The incubation comprises maintaining the fluid and sample under conditions favorable for a reaction, namely, the capture of an analyte by the functionalized MNPs. For example, the fast MPS technique and device may include increasing a temperature of the fluid to an incubation temperature, which may be substantially the same as a physiological temperature, for an incubation time period. Additionally or alternatively, the fast MPS technique or device may include agitating the fluid and sample for the incubation time period. Agitating the sample and fluid may increase the motion of the contents of the fluid and sample, and increase a rate at which analytes interact or contact functionalized MNPs, thereby increasing a rate of capture of the analytes by the MNPs (e.g., by MNPs functionalized to comprise probes configured to capture analytes). Increasing the temperature of the fluid and sample may also increase the motion of the contents of the fluid and sample, and may also increase a reactivity between the functionalization of the MNPs (e.g., probes) and analytes, thereby also increasing the rate of capture of the analytes by the MNPs (e.g., probes).

Accordingly, the techniques disclosed may provide one or more technical advantages. For example, the techniques and devices described herein may reduce an assay time, and may provide a rapid, convenient, and widely deployable diagnostic tool for surveillance of diseases and/or conditions, which may contribute to early detection and treatment, as well as to mitigating the spread of the disease and/or condition within a community and/or across communities.

In one example, this disclosure describes a method including: receiving a sample in a fluid, the fluid comprising a plurality of surface functionalized magnetic nanoparticles (MNPs), wherein the surface functionalized MNPs comprise a probe configured to capture an analyte; increasing a rate of capture by which the probe is configured to capture the analyte within the fluid; and subsequent to increasing the rate of capture, sensing, via volumetric-based magnetic particle spectroscopy (MPS), a magnetic response of the plurality of surface functionalized MNPs, wherein the magnetic response is indicative of whether the sample comprises the analyte.

In another example, this disclosure describes a bioassay system including: a volumetric-based magnetic particle spectroscopy (MPS) device configured to determine a magnetic response indicative of whether a sample comprises an analyte; a fluid comprising a plurality of surface-functionalized magnetic nanoparticles (MNPs), wherein the fluid is configured to receive the sample, wherein the surface functionalized MNPs comprise a probe configured to capture the analyte; and an incubator configured to increase a rate of capture by which the probe is configured to capture the analyte within the fluid subsequent to the fluid receiving the sample, wherein the volumetric-based MPS device is configured to determine the magnetic response subsequent to increasing the rate of capture.

In another example, this disclosure describes a volumetric-based magnetic particle spectroscopy (MPS) device including: at least one conductive excitation coil, the at least one conductive excitation coil configured to generate an alternating magnetic field including a plurality of frequencies; a sample mount configured to position a fluid within the at least one conductive excitation coil, the fluid comprising a plurality of surface-functionalized magnetic nanoparticles (MNPs), wherein the fluid is configured to receive a sample, wherein the surface functionalized MNPs comprise a probe configured to capture an analyte; an incubator configured to increase a rate of capture by which the probe is configured to capture the analyte; at least one sensing conductive coil configured to determine a magnetic response of the fluid positioned within the sample mount to the alternating magnetic field subsequent to increasing the rate of capture; processing circuitry configured to determine whether the sample comprises the analyte based on the magnetic response. The details of one or more examples are set forth in the accompanying drawings and the description below. Other features, objects, and advantages will be apparent from the description, drawings, and claims.

### BRIEF DESCRIPTION OF THE DRAWINGS

FIG. 1 is an illustration of an example diagnosis system including an MPS handheld device, in accordance with one or more techniques of this disclosure.

FIG. 2 is a block diagram of example circuit of an example MPS handheld device, in accordance with one or more techniques of this disclosure.

FIG. 3A is a block diagram of another example circuitry of an example MPS handheld device, in accordance with one or more techniques of this disclosure.

FIG. 3B is a conceptual diagram illustrating an example a two-stage lock-in MPS system and signal flow, in accordance with one or more techniques of this disclosure.

FIG. 4A is a schematic cross-sectional illustration of a portion of an example MPS handheld device, in accordance with one or more techniques of this disclosure.

FIG. 4B is an illustration of an example plot of the amplitude of a composite excitation magnetic field as a function of frequency, in accordance with one or more techniques of this disclosure.

FIG. 4C is an illustration of an example plot of the amplitude of magnetic responses from MNPs detected by pick-up coils as a function of frequency, in accordance with one or more techniques of this disclosure.

FIG. 5A is an illustration an example plot of the amplitude of an alternating magnetic field generated by drive coils as a function of time, in accordance with one or more techniques of this disclosure.

FIG. 5B is an illustration an example plot of the amplitude of composite magnetic field as a function of frequency, in accordance with one or more techniques of this disclosure.

FIG. 5C is an illustration of an example plot of the magnetic response of MNPs as a function of magnetic field, in accordance with one or more techniques of this disclosure.

FIG. 5D is an illustration an example plot of the amplitude of a magnetic response of one or more MNPs within the generated magnetic field of FIG. 5A as a function of time, in accordance with one or more techniques of this disclosure.

FIG. 5E is an illustration of an example plot of the amplitude of the magnetic response of FIG. 5D as a function of frequency, in accordance with one or more techniques of this disclosure.

FIG. 6A is an illustration of an example plot of the harmonic amplitude response as a function of hydrodynamic size of MNPs, in accordance with one or more techniques of this disclosure.

FIG. 6B is an illustration of an example Neel motion of an MNP, in accordance with one or more techniques of this disclosure.

FIG. 6C is an illustration of an example Brownian motion of an MNP, in accordance with one or more techniques of this disclosure.

FIG. 7 is a flowchart of an example method of measuring a sample using MPS handheld device, in accordance with one or more techniques of this disclosure.

FIG. 8 is an illustration of incubation conditions applied for reduction of magnetic particle spectroscopy (MPS) bioassay time, in accordance with one or more techniques of this disclosure.

FIG. 9 includes graphs illustrating MPS reading recorded from samples that have undergone different incubation conditions categorized by incubation temperatures of 25° C., 32° C., 37° C., and 42° C., in accordance with one or more techniques of this disclosure.

FIG. 10 is a histogram of third harmonic amplitude at different temperatures, in accordance with one or more techniques of this disclosure.

FIG. 11 is a graph illustrating a concentration-response curve of SARS-CoV-2 spike protein, in accordance with one or more techniques of this disclosure.

FIG. 12 is a graph illustrating MPS for detection of influenza A virus H1N1, in accordance with one or more techniques of this disclosure.

FIG. 13 includes graphs illustrating size distribution (DLS) with corresponding TEM (transmission electron microscopy) images, in accordance with one or more techniques of this disclosure.

FIG. 14 includes graphs illustrating SARS-CoV-2 spike protein concentration and SARS-CoV-2 spike protein amount, in accordance with one or more techniques of this disclosure.

### DETAILED DESCRIPTION

In recent years, magnetic particle spectroscopy (MPS) has emerged as a new technology for immunoassay applications. In MPS, alternating magnetic fields may be applied to magnetic nanoparticles (MNPs). The magnetic responses of these nanoparticles may be collected and recorded by a pair of specially designed pick-up coils. These magnetic responses may contain higher harmonics that are specific to the physical changes of the nanoparticles, such as binding events of target analytes to nanoparticles. MPS may be a volumetric-based bioassay method that analyzes the response signal from the whole nanoparticle suspension. In examples, a handheld MPS system that may have high, or increased, sensitivity, reduced cost, may be performed in vitro, and may be an easy-to-use point-of-care (POC) detection kit is disclosed. Examples of handheld MPS systems are described in WO 2021/212144 entitled “MAGNETIC PARTICLE SPECTROSCOPY METHOD AND DEVICE,” the contents of which are incorporated by reference herein.

With the coronavirus disease 2019 (COVID-19) pandemic, it is desirable to have a rapid, convenient, and widely deployable diagnosis tools for the surveillance of severe acute respiratory syndrome-coronavirus-2 (SARS-CoV-2), to mitigate its spread within and across communities. Magnetic particle spectroscopy (MPS) is an emerging bioassay platform that has been used extensively in the areas of oncology, food safety, bacteria and virus detection. Although it has been reported that portable MPS devices with low assay costs and easy-to-use features can be used for potential field testing, these MPS systems share the same drawback with most point-of-care (POC) diagnosis techniques that is the long bioassay time.

For example, the fastest on-site detection techniques such as lateral flow assays and ID Now™ by Abbot Point of Care, Inc. can detect COVID-19 in 15-30 min. For most on-site diagnosis platforms, the bioassay time varies from 1 hour to 12 hours. As a result, the long turnaround time has severely hindered the COVID-19 surveillance and impeded pandemic control measures. This disclosure describes a MPS bioassay strategy that may reduce assay time (e.g., to about 5 minutes). In one or more examples, surface functionalized magnetic nanoparticles (MNPs) are incubated with target analytes at 37° C. with agitation for a time period on the order of minutes, e.g., 3 minutes, and the MPS reading is then taken relatively shortly thereafter, e.g., at the 5^{th} minute. By using a volumetric MPS bioassay platform as a model, the example techniques may show feasibility of an ultra-fast (e.g., 5-minute) detection of SARS-CoV-2 spike protein with a detection limit below 5 nanomoles (nM), e.g., 0.2 picomoles (pmole). The example techniques for a fast bioassay strategy, e.g., a “5-minute bioassay” strategy, may be applied to reduce assay time for other liquid phase, volumetric biosensors such as nuclear magnetic resonance (NMR), quantum dots (QD), fluorescent biosensors, etc. Although the disclosure may refer to the example techniques as 5-minute bioassay, the example techniques should not be considered as requiring completion in 5 minutes. The “5-minute bioassay” phrase is used simply as an example.

Coronavirus disease 2019 (COVID-19) pandemic is caused by severe acute respiratory syndrome coronavirus-2 (SARS-CoV-2) and is associated with severe respiratory distress. A rapid and sensitive method for early detection of SARS-CoV-2 is useful for controlling the spread of the COVID-19 pandemic by proper containment procedures as well as for reducing morbidity and mortality by facilitating early treatment. Magnetic particle spectroscopy (MPS) was originally derived from magnetic particle imaging (MPI), and is used as a bioassay technique. There may be two types of MPS bioassay platforms, namely, the surface- and volumetric-based MPS bioassays.

Both platforms rely on monitoring the dynamic magnetic responses of magnetic nanoparticles (MNPs), but may use different mechanisms. Surface-based MPS bioassay platforms are usually combined with lateral flow strips or non-magnetic porous filters that are surface functionalized to specifically capture target biochemical analytes and MNPs. This surface-based MPS bioassay strategy has been reported for the detection of SARS-CoV-2, plant viruses, toxins, and drugs.

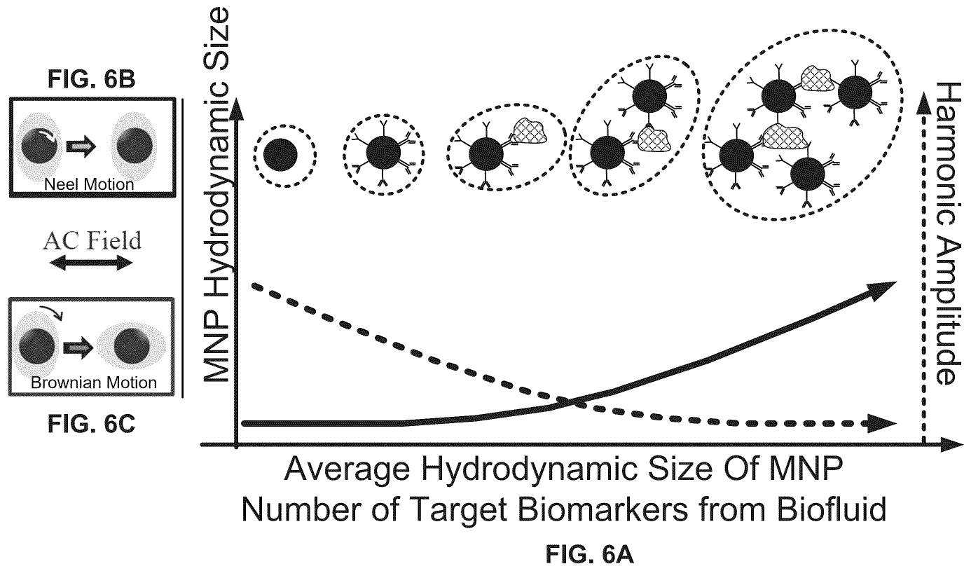

The volumetric-based MPS bioassay quantifies biochemical analytes through the change of dynamic magnetic responses of freely rotating MNPs before and after the specific binding events. By using specially designed, surface functionalized MNPs, the presence of target biochemical analytes causes different degrees of MNP clustering (FIG. 6A), which impedes the Brownian relaxation of MNPs under an AC magnetic field (FIG. 6B). Thus, weaker dynamic magnetic responses and lower harmonic amplitudes (i.e., MPS spectra) are observed. This volumetric-based MPS bioassay strategy may be used for the detection of SARS-CoV-2, H1N1 virus, thrombin and DNA aptamers, hormones and cytokines.

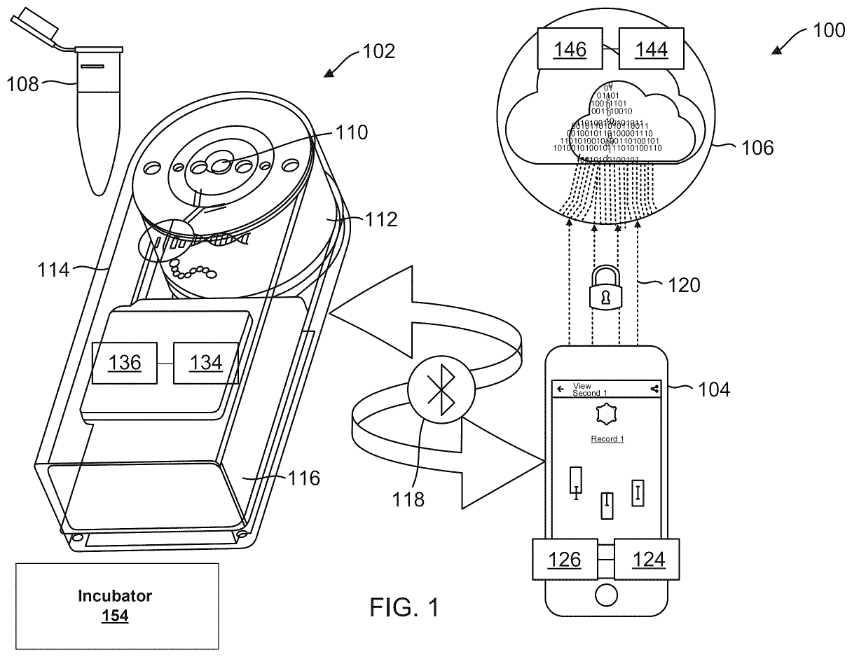

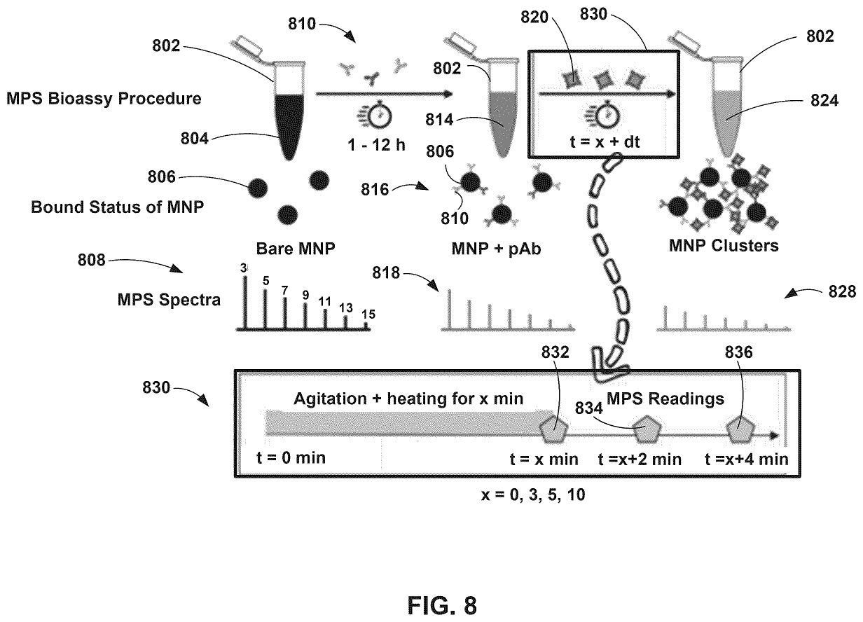

Compared to surface-based MPS bioassay, this homogeneous and volumetric MPS bioassay strategy may be easily adapted into a one-step, wash-free testing kit for on-site applications, due to its ease of use. The end users may simply mix the surface functionalized MNPs with the liquid sample and take MPS readings. However, the bioassay step usually takes 1 hour to 12 hours until the specific binding stabilizes at equilibrium (FIG. 8). This delay is a major obstacle to the transfer of volumetric MPS bioassays from lab to field testing. For instance, FIG. 1 illustrates incubation conditions applied in order to reduce the MPS bioassay time, (i) MNP surface functionalization with polyclonal antibodies, and (ii) surface functionalized MNPs incubating with target analytes.

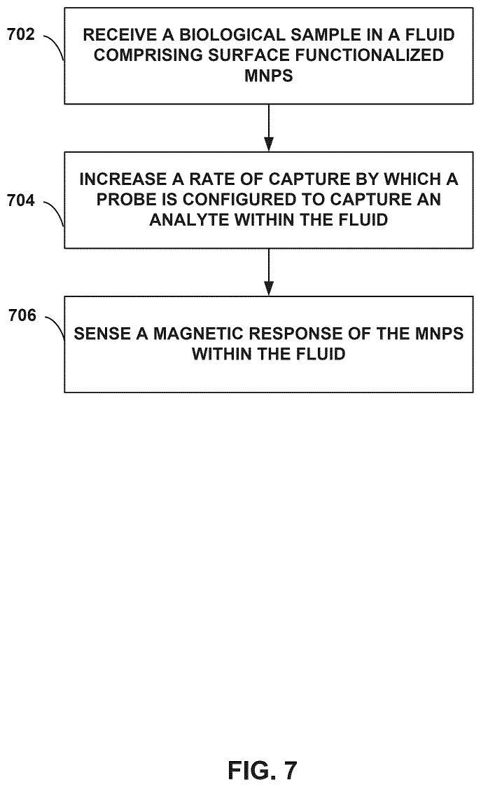

This disclosure describes examples of the possibility of reducing the bioassay time by heating and agitating samples, e.g., incubating (e.g., FIG. 7). The thermal energy and vibrational kinetic energy (caused by agitation) may increase the frequency of successful collisions between capture probes, e.g., polyclonal antibodies (pAb) in some examples, and target analytes, e.g., SARS-CoV-2 spike protein in some examples, which may provide for faster specific binding and shorter diagnosis turnaround time.

FIG. 1 is an illustration of an example diagnosis system 100 including an MPS handheld device 102, in accordance with one or more techniques of this disclosure. In the example shown, diagnosis system 100 includes MPS handheld device 102, a computing device 104, a distributed computing system 106, e.g., cloud computing system 106, a sample vial 108, and an incubator 154.

In the example shown, MPS handheld device 102 includes a sample loading port 110, coils 112, a housing 114, and circuitry 116. Sample loading port 110 may be configured to accept samples, for example, sample vial 108, and to position the sample to be tested in the correct position with respect to coils 112 for an MPS measurement or measurements. Coils 112 may include drive coils, e.g., primary coils, secondary coils, etc., and pick-up coils, or any type of coil suitable for forming, controlling, and detecting or sensing magnetic fields. Coils 112 may be made of any suitable conductive and/or magnetic material, for example, copper, silver, aluminum, or the like. Housing 114 may be configured to enclose, support, and position the components of MPS handheld device 102 correctly with respect to each other, e.g., sample loading port 110 and coils 112, to provide a structure for connecting circuitry 116 to coils 112 and any other components of MPS handheld device 102, and to provide structure for a user physically manipulate MPS handheld device 102. In some examples, housing 114 may be 3D printed with any suitable material, such as a polymer. In some examples, the polymer may include polylactic acid (PLA). In some examples, MPS handheld device 102 may be manipulatable by hand by a user, e.g., MPS handheld device 102 may be a handheld device.

In some examples, sample vial 108 may include or contain a liquid including one or more MNPs. In some examples, material to be tested, e.g., a fluid such as a sample of a patient's blood or blood components, may be added to sample vial 108. The MNPs included in sample vial 108 may be surface functionalized via coating with ligands (e.g., carboxylic acid and amine, and the like), proteins (e.g., antibodies, polyclonal antibodies, streptavidin, protein A, and the like), antigens, nucleic acids (e.g., deoxyribonucleic acid (DNA), ribonucleic acid (RNA), or the like) or any combination thereof, or may be carried in a liquid. Sample vial 108 may be configured to have a long shelf life, for example, a shelf life greater than one hour, greater than one day, greater than one week, greater than one month, greater than one year, greater than ten years, or any other long shelf life. In some examples, the MNPs included in sample vial 108 may be configured to be stored at room temperature, or at lower temperatures, for example, near 4° Celsius (C). The MNPs included in sample vial 108 may be configured to be surface functionalized with different capture probes, e.g., antibodies, antigens, DNA, RNA, and the like, designed to detect one or more specific biomarkers, e.g., one or more specific disease. In some examples, sample vial 108 may be a flat bottom, USP type I glass vial, may have dimensions of 31 millimeters (mm) by 5 mm and a volume capacity of 0.25 milliliters (mL), and may be one-time use only, e.g., disposable. In other examples, sample vial 108 may be a plastic vial, or made of any other suitable material.

In some examples, MPS handheld device 102 may be communicatively coupled, for example by a wired or a wireless connection 118 and/or 120, to computing device 104 and/or distributed system 106. In some examples, connection 118 and/or 120 may be a secured connection, e.g., encrypted, requiring two-factor authentication, and the like. Measurements and/or information corresponding to measurements may be transferred to computing device 106 and/or distributed system 106, for example, for processing of measurements and/or information corresponding to measurements. In some examples, circuitry 116 of MPS handheld device 102 may include processing circuitry 136 and memory 134, and may process measurements and/or information corresponding to measurements without transferring the measurements and/or information corresponding to measurements to computing device 104 or distributed computing system 106. In some examples, computing device 104 may be communicatively coupled to distributed computing system 106, for example by a wired or a wireless connection 120, and measurements and/or information corresponding to measurements from MPS handheld device 102 received by computing device 104 may be transferred to distributed computing system 106, for example, for processing of measurements and/or information corresponding to measurements.

In the illustrated example, computing device 106 may include processing circuitry 126 coupled to memory 124 and to a display, one or more outputs, and one or more user inputs of a user interface. Processing circuitry 126, as well as processing circuitry 136, and other processing modules or circuitry described herein, e.g., processing circuitry 146 of distributed computing system 106, may be any suitable software, firmware, hardware, or combination thereof. Processing circuitry 126, 136, 146 may include any one or more microprocessors, controllers, digital signal processors (DSPs), application specific integrated circuits (ASICs), field-programmable gate arrays (FPGAs), or discrete logic circuitry. The functions attributed to processors described herein, including processing circuitry 126, may be provided by processing circuitry of a hardware device, e.g., as supported by software and/or firmware.

In some examples, processing circuitry 126, as well as processing circuitry 136, 146, may be configured to determine diagnosis information associated with MPS measurements and/or MPS measurement information. For example, the processing circuitry 126 may determine amplitudes and/or phases of magnetic fields detected via coils 112 and may perform any suitable signal processing to determine a diagnosis based on the amplitudes and/or phases of the magnetic fields. Processing circuitry 126 may also receive input signals from additional sources (not shown). For example, processing circuitry 126 may receive an input signal containing position information, such as Global Navigation Satellite System (GNSS) coordinates of MPS handheld device 102 and/or computing device 104. Additional input signals may be used by processing circuitry 126 in any of the calculations or operations it performs. In some examples, processing circuitry 126 may be adapted to execute software, which may include an operating system and one or more applications, as part of performing the functions described herein. In some examples, processing circuitry 126 may include one or more processing circuitry modules for performing each or any combination of the functions described herein.

In some examples, processing circuitry 126 may be coupled to memory 124, processing circuitry 136 may be coupled to memory 134, and processing circuitry 146 may be coupled to memory 144. Memory 124, as well as memory 134 and 144, may include any volatile or non-volatile media, such as a random-access memory (RAM), read only memory (ROM), non-volatile RAM (NVRAM), electrically erasable programmable ROM (EEPROM), flash memory, and the like. Memory 124, 134, and 144 may be a storage device or other non-transitory medium. Memory 124, 134, and 144 may be used by processing circuitry 126, 136, and 146, respectively, for example, to store information corresponding MPS handheld device 102 measurements. In some examples, processing circuitry 126, 136, and 146 may store measurements or previously received data in memory 124, 134, and 144, respectively, and/or calculated values for later retrieval. In some examples, MPS handheld device 102 may be powered by a wall-plug, e.g., via alternating current (AC) power and may include power circuitry such as an AC adapter. In some examples, MPS handheld device 102 may be alternatively and/or additionally powered by batteries, solar cells, or any other suitable power source.

Processing circuitry 126 may be coupled to a user interface including a display, user inputs, and outputs. In some examples, the display may include one or more display devices (e.g., monitor, personal digital assistant (PDA), mobile phone, tablet computer, any other suitable display device, or any combination thereof). For example, the display may be configured to display measurements, measurement information, and/or diagnosis information. In some examples, the user input is configured to receive input from a user, e.g., information corresponding to MPS handheld device 102, a patient, and/or a sample, e.g., sample vial 108. For example, a user may input information such as MPS handheld device 102 parameters or sample vial 108 information by manually entering the product number from MPS handheld device 102 or sample vial 108, or by scanning a quick response (QR) code/bar code from MPS handheld device 102 or sample vial 108. In some examples, MPS handheld device 102 and/or sample vial 108 may be labeled with a serial number as well as a QR code or bar code for a user to input information before testing.

The user input may include components for interaction with a user, such as a keypad and a display, which may be the same as the display. In some examples, the display may be a cathode ray tube (CRT) display, a liquid crystal display (LCD) or light emitting diode (LED) display and the keypad may take the form of an alphanumeric keypad or a reduced set of keys associated with particular functions. The user input, additionally or alternatively, may include a peripheral pointing device, e.g., a mouse, via which a user may interact with the user interface. In some examples, the displays may include a touch screen display, and a user may interact with the user input via the touch screens of the displays. In some examples, the user may also interact with the user input remotely via a networked computing device.

Incubator 154 is configured to increase a rate of capture by which probe of an MNP may capture an analyte. For example, incubator 154 may be configured to maintain a temperature of a sample within sample vial 108 (or sample vial 108 itself) after a material to be tested (e.g., a blood sample) is added to sample vial 108, for an incubation time period. Incubator 154 may be configured to maintain an incubation temperature, which may be a temperature elevated from a room temperature, and which may be a physiological temperature, or a body temperature (e.g., of the patient or animal from which the material to be tested is taken). For example, incubator 154 may be configured to maintain a temperature of greater than or equal to 25 degrees Celsius (° C.) and less than or equal to 42° C., e.g., a physiological temperature, or body temperature, of a human of about 37° C., for an incubation time of greater than or equal to 1 minute, or for any suitable incubation time period.

In some examples, incubator 154 is configured to agitate the sample (e.g., within sample vial 108) for the incubation time period, e.g., at room temperature or while maintaining the incubation temperature. For example, incubator 154 may be configured to shake or stir the sample. In other examples, incubator 154 may be configured to apply a rotating magnetic field to sample vial 108, causing the MNPs to move within the sample and agitate the sample.

In the example shown, incubator 154 is a separate device from MPS handheld device 102. In some examples, a user may add a biological sample to a fluid containing surface functionalized MNPs within vial 108, and incubate the fluid including the sample via incubator 154 before placing vial 108 in loading port 110 and taking a measurement. In other examples, incubator 154 may be a part of MPS handheld device 102. For example, MPS handheld device 102 may be configured to incubate the fluid including the sample when vial 108 is placed within loading port 110, e.g., before or during taking a measurement. For example, MPS handheld device 102 may include a heater, a motion-inducing component, and/or coils configured to apply a rotating magnetic field to the fluid including the MNPs and biological sample to incubate the sample to be tested. In some examples, coils 112 may be configured to incubate the sample within sample vial 108, e.g., apply a rotating magnetic field to agitate the fluid within sample vial 108, as well as form, control, detect, or sense magnetic fields for MPS measurements.

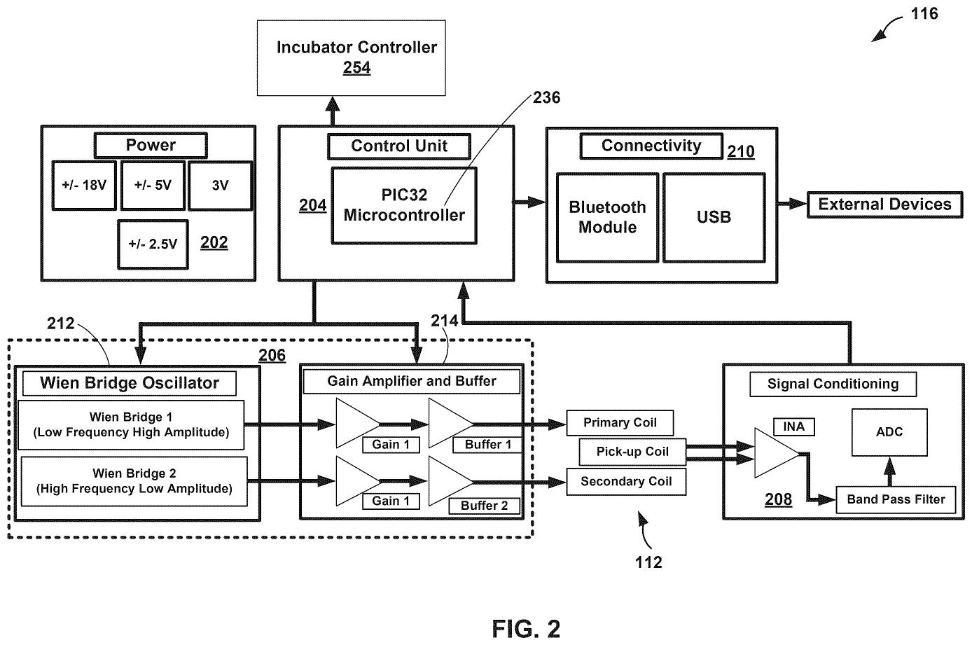

FIG. 2 is a block diagram of example circuitry 116 of an example MPS handheld device 102, in accordance with one or more techniques of this disclosure. In the example shown, circuitry 116 includes power unit 202, control unit 204, coil driver 206, signal conditioning unit 208, connectivity unit 210, coils 112, and incubator 254.

In the examples shown, power unit 202 is configured to generate electrical power, for example, usable direct current (DC) voltages, from an off-board alternating current (AC) power supply to be used by multiple digital and analog system components. Power unit 202 may comprise an off-board AC to DC power supply adapter to provide DC power to MPS handheld device 102. In some examples, the DC voltage may be further dropped down using high current rated linear drop-off (LDO) regulators and switching power supply components for providing a stable supply at suitable voltages to be used by different stages of MPS handheld device 102. For example, an LDO may be utilized to generate a +/−2.5V supply voltage, or a +/−5V supply voltage, or a +3.3V supply voltage, or any other suitable supply voltage to power an onboard microcontroller. In some examples, switching regulators may be used to generate −15V and +15V supply voltages to feed the coils 112, as described further below. In other examples, power unit 202 may include a battery or other portable power source and associated voltage regulation circuitry.

In some examples, control unit 204 may include microcontroller 236. In some examples, microcontroller 236 may include built-in floating-point hardware and may be utilized as an on-board processor, e.g., as processing circuitry 136. In some examples, microcontroller 236 may communicate with an analog-to-digital converter (ADC) and a communication connection module, e.g., using a using a serial peripheral interface (SPI) protocol. Microcontroller 236 may be additionally configured to select variable frequencies for coils 112, e.g., the primary and secondary drive coils, via SPI protocol to communicate with digital potentiometers for selecting appropriate excitation frequencies.

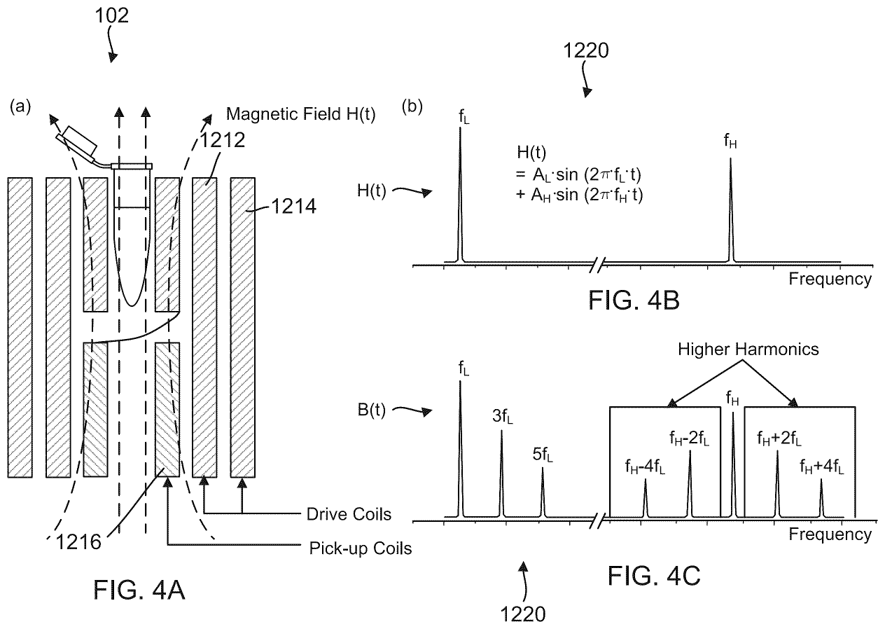

In the example shown, coil driver 206 may be configured to generate variable frequency waveforms for driving coils of coils 112. For example, coil driver 206 may generate two waveforms for primary and secondary drive coil excitation. In some examples, coil driver 206 may generate variable frequency waveforms via two sub-stage units, e.g., Wien-Bridge Oscillator 212 and gain amplifier and buffer 214. Wien-Bridge Oscillator 212 may generate base waveform signals to be further processed by later stages. Gain amplifier and buffer 214 may amplify incoming waveforms and may provide a buffer to meet high current requirements of coils 112. In some examples, waveforms may include any of a sinusoidal waveform, a rectangular or square waveform, a triangular waveform, a sawtooth waveform, or any combination thereof. In some examples, Wien-Bridge Oscillator 212 and gain amplifier and buffer 214 may include corresponding sets of circuitries for generating a low frequency high amplitude waveform for output to a primary coil and for generating a high frequency low amplitude waveform for output to a secondary coil. For example, MPS handheld device 102 may include primary coils 112 (e.g., which may be drive coils 1212 illustrated and described below with reference to FIG. 4A) comprising 1278 turns of 13 American Wire Gage (AWG) copper wire for the low frequency high amplitude waveform, secondary coils 112 (e.g., drive coils 1214 of FIG. 4A below) comprising 449 turns of 30 AWG copper wire for the high frequency low amplitude waveform, and pick-up coils 112 (e.g., pick-up coils 1216 of FIG. 4A below) comprising 36AWG copper wire.

In some examples, coil driver 206 driving coils of coils 112 may be configured to generate phase stable magnetic fields.

In the example shown, signal conditioning unit 208 may be configured to remove (e.g., filter) noise and amplify a signal received from pick-up coils of coils 112. For example, one or more pick-up coils of coils 112 may generate a differential voltage output, and signal conditioning unit 208 may condition the differential voltage output to remove noise and amplify the differential output. In some examples, signal conditioning unit 208 may provide an initial gain and convert the differential signal from pick-up (e.g., search) coils to a single-ended signal for further processing by filtering stages. In some examples, signal conditioning unit 208 may be configured to provide Sallen-Key based second-order low-pass and high-pass filtering to remove the powerline and high-frequency noises. In some examples, a cutoff frequency of a band-pass filter may be set at or near 53 kilohertz (kHz) for the low pass filtering, e.g., as the high frequency cutoff, and at or near 730 Hz for high pass filtering, e.g., as the low frequency cutoff. The filtered signal may be sampled at, for example, 200 kilo-samples per second (ksps) having 16-bit samples via an analog-to-digital (ADC) in communication with microcontroller 236 using SPI protocol. In some implementations, microcontroller 236 may transmit data sampled by the ADC via connectivity unit 210.

In the example shown, connectivity unit 210 may be configured to transfer data via a wired or wireless connection, e.g., connection 118. In some examples, connectivity unit 210 may communicate using any wired or wireless communication modality, for example, serial, universal serial bus (USB), WiFi, local area network (LAN), Bluetooth®, and the like. In some examples, connectivity unit 210 may be configured to execute application software. For example, application software may include a user interface configured to initiate execution of processing of information via microcontroller 236 and display of the processed information in real-time. Application software may further be configured to guide users on how to use MPS handheld device 102. In some examples, the application software may be executed on an external device, for example, computing device 104 or distributed computing system 106, and may use information transferred from connectivity unit 210. In some examples, the application software may be compatible with one or more operating systems. e.g., Windows, iOS, Android™, or any other suitable operating system.

In some examples, application software may include a mobile application, and may implement a Fast Fourier Transform (FFT) for frequency-domain processing of incoming information, e.g., executed by processing circuitry 126, 136, and/or 146. In some examples, FFT implementation in the mobile application may be executed and provide results within seconds. In some examples, FFT implementation may result in information such as frequency harmonic amplitudes and phase information, and may be used to derive immunoassay detection. In some implementations, harmonic amplitudes, phase angle information, and harmonic ratios may be metrics for quantifying target analytes from testing samples, such as sample vial 108.

Incubator controller 254 may be configured to control incubator 154. In the example shown, microcontroller 236 is configured to communicate with incubator controller 254, e.g., in examples in which incubator 154 is integrated into MPS handheld device 102. Incubator controller 254 may comprise processing circuitry, and may be substantially similar to processing circuitry 136, or may be an example of processing circuitry 136. In other examples, incubator controller 254 may be processing circuitry that is separate from, and may or may not be in communication with microcontroller 236, e.g., in examples in which incubator 154 is separate from MPS handheld device 102.

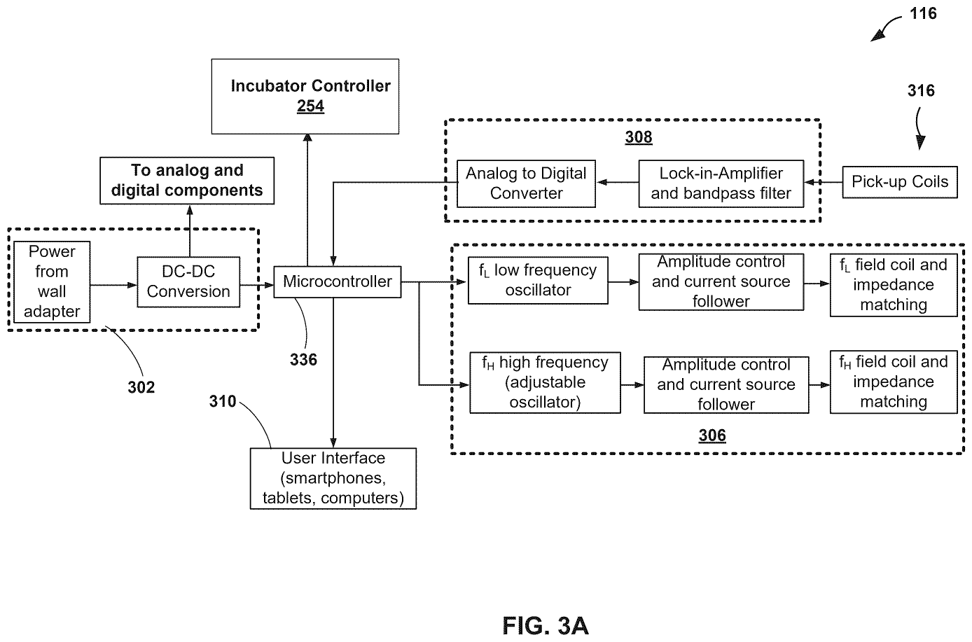

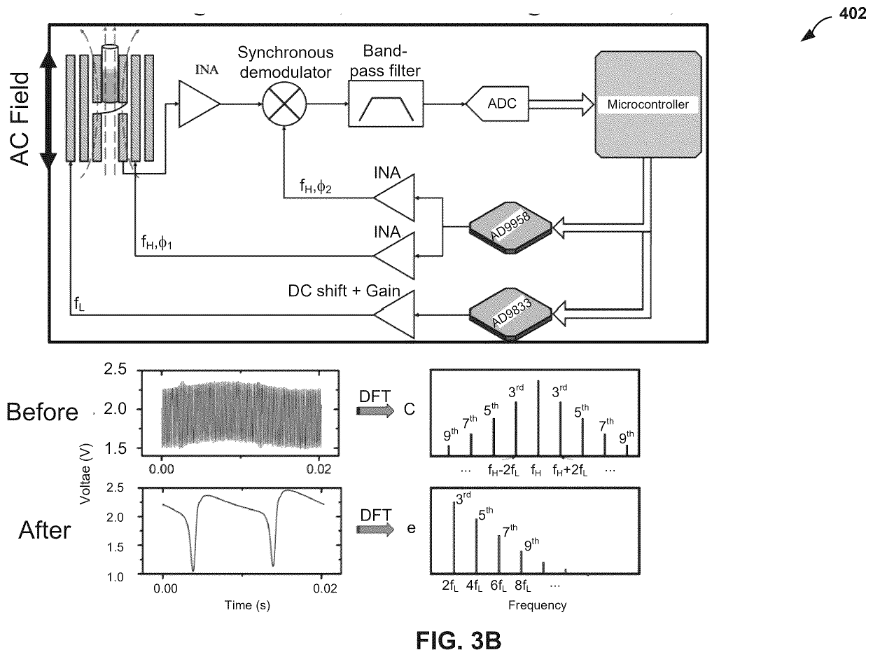

FIG. 3A is a block diagram of another example circuitry 116 of an example MPS handheld device 102, in accordance with one or more techniques of this disclosure. In the example shown, circuitry 116 includes power unit 302, microcontroller 336, coil driver 306, signal conditioning unit 308, connectivity unit 310, coils 112, and incubator controller 354.

In the examples shown, power unit 302 is configured to generate electrical power, for example, usable direct current (DC) voltages from an off-board alternating current (AC) power supply, e.g., a wall outlet. Power unit 302 may be substantially similar to power unit 202 illustrated and described above with respect to FIG. 2, and may additionally include DC to DC conversion.

Microcontroller 336 may be utilized as an on-board processor, e.g., as processing circuitry 136, and may be substantially similar to microcontroller 236 illustrated and described above with respect to FIG. 2. Incubator controller 354 may be configured to control incubator 154, and may be substantially similar to incubator controller 254 illustrated and described above with respect to FIG. 2.

In the example shown, coil driver 306 may be configured to generate variable frequency waveforms for drive coils, e.g. coils 112, and may be substantially similar to coil driver 206 illustrated and described above with respect to FIG. 2. In the examples shown, coil driver 306 may generate a low frequency waveform via a low frequency oscillator and a high frequency waveform via a high frequency oscillator. Coil driver 306 may be configured to control the amplitudes and impedance matching of the low and high frequency waveforms. In some examples, waveforms may include any of a sinusoidal waveform, a rectangular or square waveform, a triangular waveform, a sawtooth waveform, or any combination thereof.

In the example shown, signal conditioning unit 308 may be configured to remove noise and amplify a signal received from pick-up coils 316, and perform analog to digital conversion. In some examples, signal conditioning unit 308 may be substantially similar to signal conditioning unit 208. For example, one or more pick-up coils of coils 112 may generate a differential voltage output, and signal conditioning unit 308 may operate to condition the differential voltage signal similar to signal conditioning unit 208 illustrated and described above with respect to FIG. 2.

In the example shown, connectivity unit 310 may be configured to transfer data via a wired or wireless connection, e.g., connection 118. Connectivity unit 310 may be configured to provide an interface to external devices, e.g., external computing devices. In some examples, connectivity unit 310 may be substantially similar to connectivity unit 210 illustrated and described above with respect to FIG. 2.

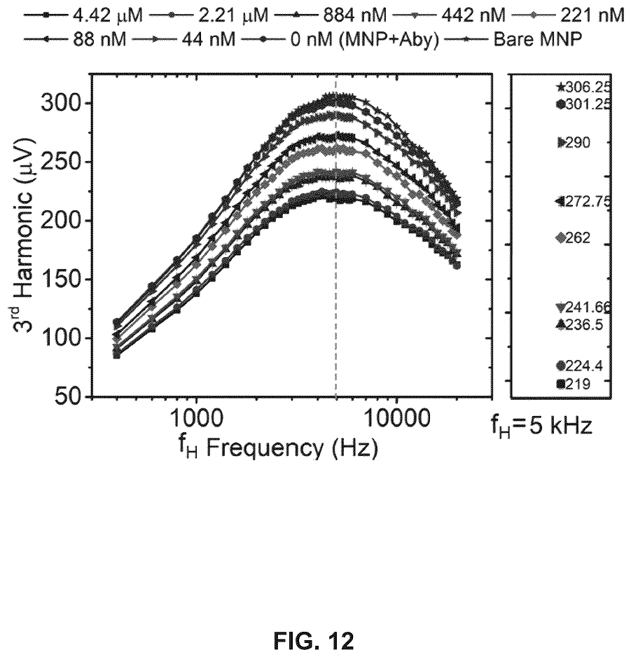

FIG. 3B is a conceptual diagram illustrating an example a two-stage lock-in MPS system 402 and signal flow, in accordance with one or more techniques of this disclosure. System 402, with an additional voltage gain for improved bioassay sensitivity, was used as part of a technique, similar to the method of FIG. 7, producing the concentration-response curve of SARS-CoV-2 spike protein illustrated in FIG. 12.

FIGS. 4A-6C illustrate one or more example operating principles of an MPS device, for example, MPS handheld device 102, and will be described concurrently below.

FIG. 4A is a schematic cross-sectional illustration of a portion of an example MPS handheld device 102, in accordance with one or more techniques of this disclosure. In the example shown, sample vial 108 is in position within sample loading port 110 in MPS handheld device 102 during a measurement, e.g., while drive coils 1212 and 1214 generate an alternating magnetic field H(t) proximate sample vial 108 and pick-up coils 1216 detect the resulting magnetic responses from MNPs within sample vial 108. Pick-up coils 1216 may be designed to have half of its portion clockwise wound and the other half portion counter-clockwise wound. This design removes the signal caused by alternating magnetic field H(t) and allows the pick-up coils 1216 to specifically detect the magnetic responses of MNPs. In some examples, drive coils 1212 may generate a magnetic field having a low frequency, f_{L}, relative to a magnetic field generated by drive coils 1214 having a higher frequency, f_{H} based on drive signals generated by processing circuitry of MPS handheld device 102. Each of drive coils 1212 and 1214 may additionally generate magnetic fields having different amplitudes, e.g., A_{L} generated via low frequency drive coils 1212 and A_{H} generated via high frequency drive coils 1214. The magnetic fields generated by drive coils 1212 and 1214 may be in phase, and may add via superposition, resulting in generation of the composite magnetic field H(t). In some examples, drive coils 1212 may be wound in an opposite direction from drive coils 1214, e.g., counter-clockwise for drive coils 1212 and clockwise for drive coils 1214, or clockwise for drive coils 1212 and counter-clockwise for drive coils 1214. In some examples, drive coils 1212 and 1214 may be wound in the same direction.

FIG. 4B is an illustration of an example plot 1220 of the amplitude of composite magnetic field H(t) as a function of frequency, in accordance with one or more techniques of this disclosure. In the example shown, drive coils 1212 and 1214 each generate a sinusoidal magnetic field, and plot 1220 illustrates the frequency content of composite magnetic field H(t), namely two substantially single frequency spikes, or delta functions, at f_{L} and f_{H} and having amplitudes A_{L} and A_{H}, respectively.

FIG. 4C is an illustration of an example plot 1250 of the amplitude of magnetic flux B(t) due to the magnetic responses of MNPs detected by pick-up coils 1216 as a function of frequency, in accordance with one or more techniques of this disclosure. In the example shown, the MNPs within sample vial 108 respond to magnetic field H(t), which may generate harmonics of H(t) that are detected by pick-up coils 1216. In the example shown, the magnetic flux B(t) including magnetic responses of MNPs may include several harmonic frequencies of varying amplitudes at various frequencies.

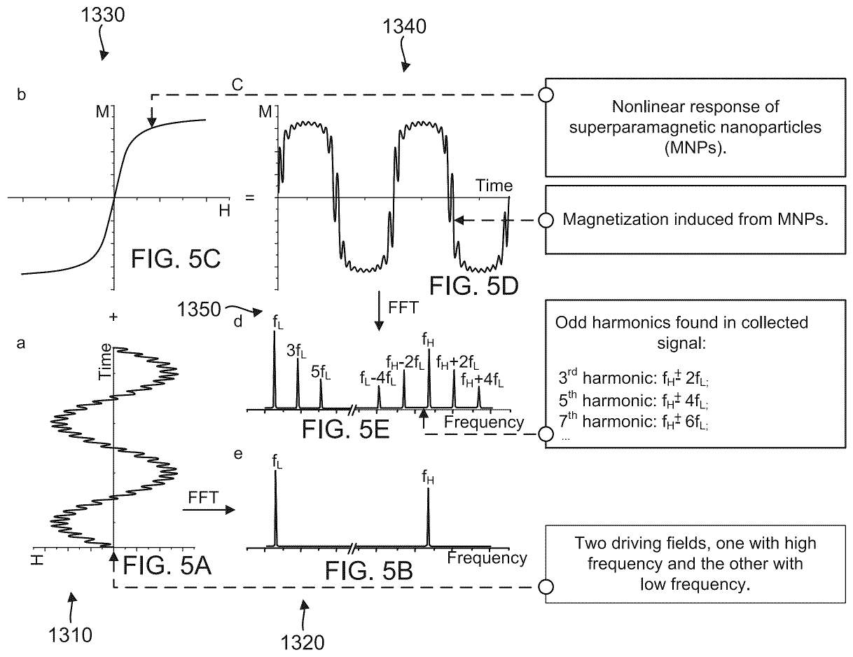

FIGS. 5A-5E illustrate the operating principle of FIGS. 4A-4C in more detail.

FIG. 5A is an illustration an example plot 1310 of the amplitude of an alternating magnetic field H(t) generated by drive coils as a function of time, in accordance with one or more techniques of this disclosure. For example, drive coils 1212 and 1214 may generate sinusoidal magnetic fields, and FIG. 5A illustrates the superposition of each low and high frequency sinusoidal magnetic field generated by drive coils 1212 and 1214, along with their corresponding differing amplitudes. FIG. 5B is an illustration an example plot 1320 of the amplitude of composite magnetic field H(t) as a function of frequency, in accordance with one or more techniques of this disclosure. In the example shown, FIG. 5B illustrates the spectral (temporal) content of H(t) of FIG. 5A, similar to FIG. 4B described above.

FIG. 5C is an illustration of an example plot 1330 of the magnetic response of MNPs to static magnetic fields (not alternating magnetic field), in accordance with one or more techniques of this disclosure. In the example shown, MNPs may by superparamagnetic, and may have a nonlinear response to an applied magnetic field.

FIG. 5E is an illustration of an example plot 1350 of the amplitude of the alternating magnetic responses of FIG. 5D as a function of frequency, in accordance with one or more techniques of this disclosure. FIG. 5D explicitly illustrates the additional harmonic content generated by the magnetic response of MNPs within sample vial 108.

In some examples, the magnetic response of MNPs within sample vial 108 may change based on the presence of analytes in a biofluid added to sample vial 108. For example, the analytes may bind to the surface functionalized MNPs and alter the magnetic response of the MNPs relative to the magnetic response of surface functionalized MNPs with no analytes present in the biofluid. In some examples, the altered magnetic response of the MNPs due to analytes may be distinguished from features of detected magnetic flux B(t), for example, via changes to the amplitudes and/or frequencies of the spectral content, e.g., harmonics, of B(t).

For example, in the presence of oscillating magnetic fields, MNPs may be magnetized and their magnetic moments may tend to align with the magnetic fields. For a ferrofluid system of monodispersed, noninteracting MNPs, the magnetic response may obey a Langevin function:

$$\begin{matrix}{M}_{D} ( t ) = {m}_{s} cL ( ξ ) , \\ where , \\ L ( ξ ) = coth ξ - \frac{1}{ξ} , ξ = \frac{{m}_{s} H ( t )}{{k}_{B} T}\end{matrix}$$

The MNPs are characterized by magnetic core diameter D, saturation magnetization M_{S} and concentration c. In some examples, MNPs may be assumed to be spherical and without mutual interactions. Consequently, the magnetic moment of each particle may be mₛ=M_{S}πD³/6, where V_{c}=πD³/6 is the volume of the magnetic core, ξ is the ratio of magnetic energy over thermal energy, k_{B} is Boltzmann's constant, and T is the absolute temperature in Kelvin. The external magnetic fields may be expressed as H(t)=A_{H} cos(2πf_{H}t)+A_{L} cos(2πf_{L}t) where A_{H}, A_{L}, f_{H}, and f_{L} are the amplitude and frequency of high and low frequency fields, respectively.

The harmonics generated by MNPs at specific frequencies may be represented by a phasor, e.g., Ae^{j(ωt+φ)}, or A∠φ, where ω is the angular frequency of the driving field, A is the harmonic amplitude, φ is the harmonic phase, and j is the square root of negative one.

FIG. 5D is an illustration of an example plot 1340 of the amplitude of an alternating magnetic responses of MNPs detected by pick-up coils 1216 as a function of time, in accordance with one or more techniques of this disclosure. The example shown in FIG. 5D illustrates the effects of the MNPs on the applied magnetic field, e.g., perturbation of H(t) resulting in generation of harmonics.

According to Faraday's law, the induced voltage in a pair of pick-up coils is expressed as:

$$u ( t ) = - {S}_{0} V \frac{d}{dt} {M}_{D} ( t )$$

where V is the volume of an MNP suspension. Pick-up coil sensitivity S₀ is equal to the external magnetic field strength divided by current.

Taylor expansion of M_{D}(t) shows the major frequency mixing components:

$$\frac{{M}_{D} ( t )}{{m}_{S} C} = L ( \frac{{m}_{s} H ( t )}{{k}_{B} T} ) = \frac{1}{3} ( \frac{{m}_{s}}{{K}_{B} T} ) H ( t ) - \frac{1}{45} {( \frac{{m}_{s}}{{k}_{B} T} )}^{3} {H ( t )}^{3} + \frac{2}{945} {( \frac{{m}_{s}}{{K}_{B} T} )}^{5} H {( t )}^{5} + … = … + [ - \frac{1}{60} {A}_{H} {{A}_{L}^{2} ( \frac{{m}_{s}}{{k}_{B} T} )}^{3} + … ] \times cos [ 2 π ( {f}_{H} \pm 2 {f}_{L} ) t + {φ}_{{f}_{H} \pm 2 {f}_{L}} ] + [ \frac{1}{1512} {A}_{H} {{A}_{L}^{4} ( \frac{{m}_{s}}{{k}_{B} T} )}^{5} + … ] \times cos [ 2 π ( {f}_{H} \pm 4 {f}_{L} ) t + {φ}_{{f}_{H} \pm 4 {f}_{L}} ] + …$$

The mixing frequency components are found at odd harmonics exclusively:

$$\begin{matrix}{M}_{D} ( t ) {\vert}_{3 rd} \approx - \frac{- {m}_{s} c}{60} {A}_{H} {{A}_{L}^{2} ( \frac{{m}_{s}}{{k}_{B} T} )}^{3} \times cos [ 2 π ( {f}_{H} + 2 {f}_{L} ) t + {φ}_{{f}_{H} \pm 2 {f}_{L}} ] \\ {M}_{D} ( t ) {\vert}_{5 th} \approx - \frac{{m}_{s} c}{1512} {A}_{H} {{A}_{L}^{4} ( \frac{{m}_{s}}{{k}_{B} T} )}^{5} \times cos [ 2 π ( {f}_{H} + 4 {f}_{L} ) t + {φ}_{{f}_{H} \pm 4 {f}_{L}} ]\end{matrix}$$

Amplitudes of induced voltages at the 3^{rd} and 5^{th} harmonics may be expressed as:

$$\begin{matrix}{u}_{3 rd} = {S}_{0} V \frac{d}{dt} {M}_{D} ( t ) {\vert}_{3 rd} \\ {u}_{5 th} = - {S}_{0} V \frac{d}{dt} {M}_{D} ( t ) {\vert}_{5 th}\end{matrix}$$

The harmonic amplitudes of the 3^{rd} and 5^{th} harmonics may be simplified as:

$$\begin{matrix}{A}_{3 rd} ∝ ( {f}_{H} \pm 2 {f}_{L} ) \times cos [ {φ}_{{f}_{H} \pm 2 {f}_{L}} ] \\ {A}_{5 th} ∝ ( {f}_{H} \pm 4 {f}_{L} ) \times cos [ {φ}_{{f}_{H} \pm 4 {f}_{L}} ] \\ where \\ {φ}_{{f}_{H} \pm 2 {f}_{L}} = arctan ( 2 π ( {f}_{H} \pm 2 {f}_{L} ) {τ}_{eff} )\end{matrix}$$

For iron oxide MNPs with diameters of 20 nanometers (nm), the effective relaxation time is dominated by Brownian relaxation:

$$τ = {τ}_{eff} = {τ}_{B 0} = \frac{3 η {V}_{h}}{{k}_{B} T}$$

In some examples, a change in MNP hydrodynamic size may cause a change in harmonic angle (phase angle), which further may cause a change in harmonic amplitude. Harmonic amplitudes may be proportional to the number of MNPs in a testing vial, and to make each testing result repeatable, a harmonic ratio of the 3^{rd} harmonic over the 5^{th} harmonic may be used to reduce and/or eliminated the effect of MNP quantities in the testing vial. The harmonic ratio of the 3^{rd} over the 5^{th} harmonics may be expressed as:

$$R 35 = \frac{{u}_{3 rd}}{{u}_{5 th}} = \frac{\frac{d}{dt} {M}_{D} ( t ) {\vert}_{3 rd}}{\frac{d}{dt} {M}_{D} ( t ) {\vert}_{5 th}} = \frac{( {f}_{H} \pm 2 {f}_{L} ) \times cos [ {φ}_{{f}_{H} \pm 2 {f}_{L}} ]}{( {f}_{H} \pm 4 {f}_{L} ) \times cos [ {φ}_{{f}_{H} \pm 4 {f}_{L}} ]}$$

In some examples, because f_{L}<<f_{H}, the harmonic ratio of the 3^{rd} over the 5^{th} may be further simplified as:

$$R 35 = \frac{cos [ {φ}_{{f}_{H} \pm 2 {f}_{L}} ]}{cos [ {φ}_{{f}_{H} \pm 4 {f}_{L}} ]}$$

In some examples, a change in MNP hydrodynamic size may cause a change in harmonic angle, which may further cause a change in the harmonic amplitude ratio.

Harmonic ratio may be used as an MNP quantity-independent parameter to monitor the binding of target analytes onto MNPs, e.g., the hydrodynamic size change. In addition, any kinds of harmonic ratios such as R37 (the 3rd over the 7th harmonic ratio), R57 (the 5th over the 7^{th} harmonic ratio), Rij (the ith over the jth harmonic ratio, where i and j are odd numbers and i≠j), or any other harmonic ratios, may be used.

FIG. 6A is an illustration of an example plot of the harmonic amplitude response as a function of hydrodynamic size of MNPs (or the number of target biomarkers from a biofluid, e.g., analytes of a biological sample), in accordance with one or more techniques of this disclosure. In the example shown, as the degree of MNP self-assembly increases in the presence of one or more analytes, the average hydrodynamic size of MNPs and MNP self-assemblies, e.g., clusters, increases, and the measured harmonic amplitudes decrease.

FIGS. 6B and 6C are illustrations of an example Neel and Brownian motions of an MNP, respectively, in accordance with one or more techniques of this disclosure. For example, as shown in FIG. 6B, Neel relaxation or motion includes reorientation of a magnetization vector inside an MNP, e.g., inside the magnetic core against an energy barrier. As shown in FIG. 6C, Brownian relaxation or motion is due to rotational diffusion of a whole particle, e.g., an MNP, or a cluster of MNPs.

FIG. 7 is a flowchart of an example method of measuring a sample using MPS handheld device, in accordance with one or more techniques of this disclosure. FIG. 8 is an illustration of incubation conditions applied for reduction of magnetic particle spectroscopy (MPS) bioassay time, in accordance with one or more techniques of this disclosure, and is described in conjunction with FIG. 7. The example method of FIG. 7 is described with respect to the diagnosis system and circuitry 116 of FIGS. 1-3B. The example method may be performed, for example, by a user interacting with incubator 154, MPS handheld device 102, and a computing device 104 and/or distributed computing device 106, executing the steps of the method. Although described with reference to incubator 154. MPS handheld device 102, and a computing device 104, the method of FIG. 7 is not so limited and may be performed with other devices, incubators, diagnostic devices (e.g., MPS, MPI, NMR, QD, fluorescent biosensors, or the like), and/or computing devices.

A fluid may receive a sample, the fluid comprising a plurality of surface functionalized MNPs, the surface functionalized MNPs comprise a probe configured to capture an analyte (702). For example, as shown in FIG. 8, a user may add a biological sample, such as a bodily fluid or tissue sample, which may or may not include analytes 820 to a fluid 814 comprising a plurality of MNPs 806 surface functionalized and comprising capture probes 810. Capture probes 810 may comprise with ligands (e.g., carboxylic acid and amine, and the like), proteins (e.g., antibodies, polyclonal antibodies, streptavidin, protein A, and the like), antigens, nucleic acids (e.g., deoxyribonucleic acid (DNA), ribonucleic acid (RNA), or the like), a peptide or any combination thereof. Analytes 820 may include a biomarker, e.g., one or more specific disease, an antigen, an antibody, a single stranded DNA, and a single stranded RNA, a heavy metal ion, a protease, human coronavirus 229E, human coronavirus OC43, SARS-CoV (2003). HCoV NL63 (2004), HKU1 (2005). MERS-CoV (2102), or SARS-CoV-2.

In the example shown, prior to adding the sample, the user may obtain and/or make a fluid 804 comprising a plurality of MNPs 806, e.g., within vial 802. The fluid, including “bare,” or non-functionalized MNPs, may have a magnetic response corresponding to MPS spectra 808, e.g., MPS handheld device 102 may measure MPS spectra 808 with fluid 804 as the sample being tested.

The user may then surface functionalize the MNPs with one or more capture probes 810, and vial 102 may comprise fluid 814 comprising a plurality of surface functionalized MNPs 816. The user may take a measurement of fluid 814, e.g., using MPS handheld device 102, which may be a baseline measurement of the magnetic response of the surface functionalized MNPs 816 within fluid 814 corresponding to MPS spectra 818. The user may then add the biological sample to fluid 814 at method step (702).

The user, or MPS handheld device 102, or incubator 154, may increase a rate by which probes 810 are configured to capture analytes 820 within fluid 814 (704). As shown in FIG. 8, the user may prepare 830 fluid 814 including the sample for a period of time (e.g., “x”+“dt”) before measurement, which may include incubating fluid 814 including the sample for an incubation time period, e.g., time period “x.” For example, after adding the sample to the fluid, the user may place vial 802 including fluid 814 including the sample into incubator 154. Incubator 154 may increase the temperature of fluid 814 and the sample to an incubation temperature for an incubation time period “x” and/or agitate fluid 814 including the sample for the incubation time period.

For example, the user may cause incubator 154 to begin incubation at time t=0, e.g., by adjusting settings of incubator 154 and initiating incubation via a user interface of computing device 104. Computing device 104 may then cause incubator 154 to incubate fluid 814 including the sample for an incubation time period “x,” e.g., which may be seconds, or minutes. In the example shown, incubator 154 incubates fluid 814 including the sample for x=3 minutes, followed by a stabilization period dt=2 minutes. In some examples, the stabilization period “dt” may allow the fluid 814 to cool down, or settle via reducing motion, before measuring the fluid.

In some examples, incubator 154 may incubate fluid 814 including the sample at a temperature in the range of 25° C. to 42° C., 26° C. to 42° C., 27° C. to 42° C., 28° C. to 42° C., 29° C. to 42° C., 30° C. to 42° C., 31° C. to 42° C., 32° C. to 42° C., 33° C. to 42° C., 34° C. to 42° C., 35° C. to 42° C., 36° C. to 42° C., 37° C. to 42° C., 38° C. to 42° C., 39° C. to 42° C., 40° C. to 42° C., or 41° C. to 42° C., or 35° C. to 39° C., or 36° C. to 38° C., or 37° C. In some examples, incubator 154 may incubate fluid 814 including the sample to a temperature that is substantially the same as a physiological temperature of the animal or organism from which the sample was taken, e.g., which may be about 37° C. for humans. In some examples, incubator 154 may incubate fluid 814 including the sample for an incubation time period (e.g., “x”) that is in a range of 1 minute to 10 minutes, 1 minute to 9 minutes, 1 minute to 8 minutes, 1 minute to 7 minutes, 1 minute to 6 minutes, 1 minute to 5 minutes, 1 minute to 4 minutes, or 1 minute to 3 minutes, or that may be substantially equal to 3 minutes.

In some examples, incubator 154 may incubate fluid 814 including the sample by agitating fluid 814 including the sample by shaking, stirring, or applying a rotating magnetic field to fluid 814 including the sample for the incubation period. In some examples, incubator 154 may incubate fluid 814 including the sample by agitating fluid 814 including the sample at room temperature or without incubating fluid 814 including the sample at an incubation temperature, before or after incubating fluid 814 including the sample at an incubation temperature, or at the same time as incubating fluid 814 including the sample at an incubation temperature.

After preparation 830, fluid 814 including the sample may be denoted as “prepared/incubated fluid 824.” The user may place vial 802 including fluid 824 within sample loading port 110 of MPS handheld device 102 and cause MPS handheld device 102 to take a measurement of fluid 824. Subsequent to the rate by which probes 810 are configured to capture analytes 820, MPS handheld device 102 may sense a magnetic response of the plurality of surface functionalized MNPs 816, or MNP clusters 826, of fluid 824 (706). For example, MPS handheld device 102 may sense a magnetic response of fluid 824 that is indicative of whether the sample comprises one or more analytes 820. In some examples, MPS handheld device 102 may determine that the sample comprises one or more analytes based on the magnetic response and generate an output (e.g., to a user interface) indicating that the sample comprises the analyte. In other examples, MPS handheld device 102 may determine that the sample does not comprise one or more analytes based on the magnetic response and generate an output (e.g., to a user interface) indicating that the sample does not comprise the analyte.

If the sample includes analytes 820, probes 810 may capture analytes 820 and may form MNP clusters 826. MNP clusters 826 may have an increased hydrodynamic size, or effective hydrodynamic size, relative to surface functionalized MNPs 816. The increased hydrodynamic size of MNP clusters 826 may change a Brownian relaxation of the MNP clusters 826 (relative to a Brownian relaxation of surface functionalized MNPs 816 for which a baseline measurement was taken), which may change the magnetic response of the MNPs corresponding to MPS spectra 828 of fluid 824, e.g., relative to MPS spectra 818 of fluid 814. If the sample does not include analytes 820, prepared/incubated fluid 824 may not include many, or any. MNP clusters 826, and the magnetic response of fluid 824 corresponding to MPS spectra 828 may change very little, or not at all, e.g., relative to MPS spectra 818 of fluid 814.

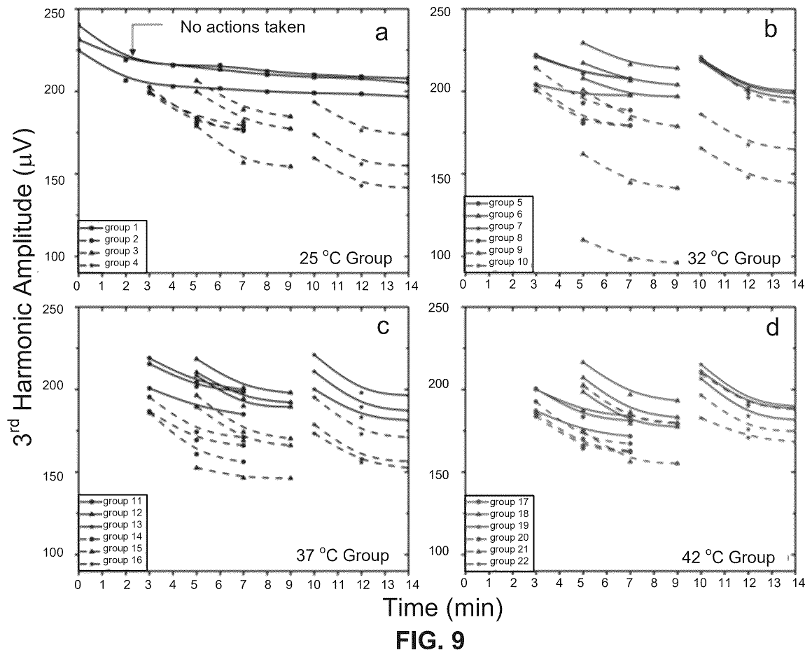

In the example shown ion FIG. 8, various incubation periods for a plurality of trials of preparing/incubating fluid 814 including the sample and measuring the resultant fluid 824. e.g., x=0 minutes (or no incubation), 3 minutes, 5 minutes, and 10 minutes, corresponding to measurement results illustrated in FIGS. 9-14.

In some examples, the method of FIG. 7 may be a wash-free, one-step bioassay method that may be performed by non-technicians with reduced and/or minimal training, and unbound target analytes may not need to be removed.

FIGS. 9-14 illustrate various measurement results using the method of FIG. 7 with various incubation parameters (e.g., incubation time, incubation temperature, and incubation agitation). Table 1 below shows three factors, or incubation parameters, corresponding to the measurement results illustrated in FIGS. 9-14. FIGS. 9-14 are described with reference to diagnosis system and circuitry 116 of FIGS. 1-3B, incubator 154, MPS handheld device 102, a computing device 104, the method of FIG. 7, and the incubation conditions illustrated in FIG. 8.

As shown in Table 1 below, the incubation time was set at 0 min (control group, no incubation), 3 min, 5 min, and 10 min. The incubation temperature was set at 25° C. (room temperature, no heating), 32° C., 37° C. (e.g., substantially a physiological temperature of humans), and 42° C., respectively, in an incubator 154. Agitation was applied by placing sample vial 802 in a Vortex Genie 2 mixer (Fisher Scientific Model G-560) set at shaker speed 2. Three independent bioassays (e.g., measurements) were carried out under each incubation condition/parameter setting. A total of 22 experimental groups were designed, where 66 samples (e.g., fluids 814 including the sample) are prepared for MPS testing/measurement.

In the examples shown, surface functionalized MNPs 816 comprised IPG30 MNPs (30 nm iron oxide nanoparticles coated with protein G, 34 nM) that were surface functionalized with anti-SARS-CoV-2 spike polyclonal antibodies (rabbit pAb. Cat: 40592-T62, Sino Biological Inc.). The MNP to pAb ratio was precisely controlled at 1:3, where theoretically each MNP is functionalized with three pAb. This ratio may be optimized based various techniques, but such optimization may not be needed. Then, 40 μL of pAb functionalized MNP complexes (denoted as ‘MNP+pAb’) was mixed with 40 μL, 10 nM SARS-CoV-2 spike protein (Cat: 40592-V08H, Sino Biological Inc.) and incubated under different conditions for x minutes (x=0, 3, 5, and 10 minutes).

Subsequently, the sample (e.g., fluid 824 within vial 802) is transferred to the MPS platform at an ambient temperature of 10° C., and three consecutive MPS readings/measurements were taken at t=x minutes, x+2 minutes, and x+4 minutes, respectively. The MPS platform consisted of a benchtop system utilizing a pair of magnetic field generation coils, one pick-up coil, data acquisition card by NI, and LabVIEW setup. Equivalently, the MPS measurements may be performed using MPS handheld device 102 described above. Taking MPS readings/measurements in an ambient temperature of 10° C. may stop the antibody-antigen binding events after x minutes of incubation and may reduce effects of elevated temperature on the MPS signal. For example, after incubating under different temperatures, samples (e.g., fluid 824 of the various trials) may be brought back to the same ambient temperature for MPS readings/measurements.

TABLE 1
Incubation condition experiment design.

| Temper- | | | | | | |
|---|---|---|---|---|---|---|
| ature | Without Agitation | | | With Agitation | | |
| 25° C. | 0 min, control group, no actions | | | 3 min | 5 min | 10 min |
| (no | taken. | | | (Group | (Group | (Group |
| heating) | (Group 1) | | | 2) | 3) | 4) |
| 32° C. | 3 min | 5 min | 10 min | 3 min | 5 min | 10 min |
| (heating) | (Group | (Group | (Group | (Group | (Group | (Group |
| | 5) | 6) | 7) | 8) | 9) | 10) |
| 37° C. | 3 min | 5 min | 10 min | 3 min | 5 min | 10 min |
| (heating) | (Group | (Group | (Group | (Group | (Group | (Group |
| | 11) | 12) | 13) | 14) | 15) | 16) |
| 42° C. | 3 min | 5 min | 10 min | 3 min | 5 min | 10 min |
| (heating) | (Group | (Group | (Group | (Group | (Group | (Group |
| | 17) | 18) | 19) | 20) | 21) | 22) |

FIG. 9 includes graphs (a)-(d) illustrating MPS readings/measurements recorded from samples (e.g., fluids 824) that have undergone different incubation conditions categorized by incubation temperatures of 25° C., 32° C., 37° C., and 42° C., in accordance with one or more techniques of this disclosure. FIG. 9 summarizes the three consecutive MPS readings/measurements of the amplitude of the 3^{rd} harmonic (in microvolts) from samples subjected to the different incubation conditions, categorized by the incubation temperatures. In some examples, higher harmonics such as the 5th, 7th, 9th, etc., show similar trends.

For example. FIG. 9 illustrates MPS readings of the 3^{rd} harmonic amplitude recorded from samples (e.g., fluids 824) that have undergone different incubation conditions, categorized by incubation temperatures, e.g., graph (a) illustrates readings after incubation at 25° C., graph (b) illustrates readings after incubation at 32° C. graph (c) illustrates readings after incubation at 37° C., and graph (d) illustrates readings after incubation at 42° C. The first data point of each curve in the respective graphs indicates the incubation time. Solid and dashed lines indicate readings of samples (e.g., fluids 824) corresponding to without agitation and with agitation, respectively. The bottom outlier in Group 9 in graph (b) (32° C. incubation example graph) is caused by air bubbles introduced into the vial during the incubation step. This outlier can be removed before further data analysis.

In the example shown in FIG. 9, control group 1 (solid lines in the graph (a) 25° C. example), illustrates readings where no actions are taken during the incubation step (e.g., no incubation). Graph (a) illustrates that the 3^{rd} harmonic amplitude control group 1 drops slowly over the 14 minutes MPS reading window. For example, each target SARS-CoV-2 spike protein molecule may host multiple distinct epitopes that provide specific binding sites for pAb, and the presence of target analytes (spike protein) may cause the cross-linking of MNPs and hinders the Brownian motion of MNPs as well as weakens the dynamic magnetic response (e.g., the amplitudes of the harmonics plotted in MPS spectra 828 may be less than the amplitudes of the harmonics plotted in MPS spectra 818). The drop in real-time harmonic amplitude may indicate that antibody-antigen specific binding is taking place, but at a slow rate.

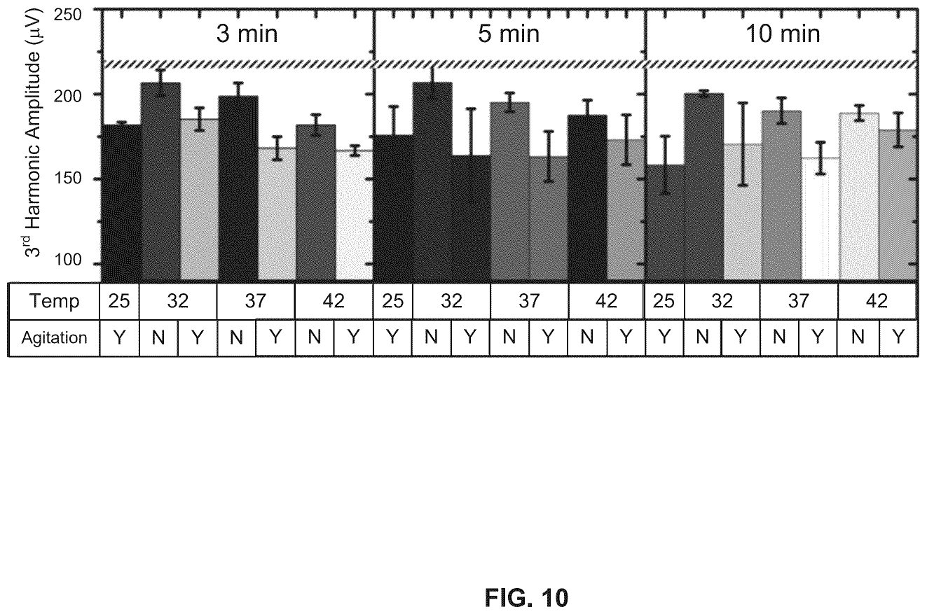

In some examples, lower harmonic amplitudes indicate higher degrees of MNP clustering with more binding events. In the graphs shown in FIG. 9, readings represented by the dashed lines (with agitation) show lower amplitudes than the solid lines for all temperature groups (a)-(d). For example, for the same incubation time and temperature, agitation may effectively accelerate antibody-antigen binding. Since all the samples are tested at an ambient temperature of 10° C., the sudden temperature drop causes a lower harmonic amplitude at t=x+2 minutes and may become stable at t=x+4 minutes (see FIG. 10). In some examples, in order to compare the heating effect in the incubation step, the first MPS readings (taken at t=x minutes) may not be used.

FIG. 10 is a histogram of the measured 3^{rd} harmonic amplitude at different temperatures corresponding to the trials of Table 1, in accordance with one or more techniques of this disclosure. FIG. 10 illustrates systematic comparison of the MPS harmonic signals from all experimental groups of Table 1. For example, FIG. 10 illustrates histograms of the 3^{rd} harmonics recorded at t=x+2 minutes. The horizontal line at y=220 μV represents the averaged 3^{rd} harmonic amplitude collected from control group 1 where no actions are taken during the incubation step, e.g., no incubation. Signals are averaged over three independent bioassays. Error bars represent standard errors. “N” and “Y” indicate without and with agitation.

As shown in FIG. 10, the 3^{rd} harmonic amplitudes are extracted from the second MPS readings (e.g., taken at t=x+2 minutes) and averaged over three independent bioassays. For all the experimental groups, the harmonic amplitudes are lower than the harmonic amplitude of control group 1 (where no actions are taken during the incubation step, marked as a horizontal line in FIG. 10). Without agitation, all experimental samples under heating conditions (e.g., at 32° C., 37° C. and 42° C.) show lower harmonic amplitudes than control group 1. In addition, for the same incubation time, a higher incubation temperature favors faster antibody-antigen binding, so lower harmonic amplitudes are observed. A longer incubation time favors more antibody-antigen binding events. When agitation is applied, heating can still accelerate antibody-antigen binding. However, if the incubation time is long (such as 5 min or 10 min), the effect of heating may become less. For example, by incubating for 3 minutes with agitation, a higher incubation temperature may favor faster binding (as observed by the lower harmonic amplitudes). However, by incubating for 5 or 10 minutes with agitation, the harmonic amplitude of the heated sample may not be significantly different from that of the unheated samples (e.g., at 25° C.).

In some examples, to effectively accelerate the binding process, incubation parameters may be set at 37° C. with agitation for 3 minutes. In some examples, reducing incubation time may be given priority, but reducing incubation time need not always be given priority. For example, although incubating at 32° C. with agitation for 5 minutes, or at 37° C. with agitation for 5 minutes, or at 25° C. with agitation for 10 minutes shows similar results, such techniques may be less favorable than incubation at 37° C. with agitation for 3 minutes in cases where reducing incubation time is given priority.

In some examples, an ultra-fast MPS bioassay strategy and/or method may include: (1) incubate samples at 37° C. with agitation for 3 minutes, (2) transfer the mixture to an ambient temperature of 10° C. (e.g., for a stabilization period “dt”), and (3) collect/measure the second MPS data point (e.g., MPS reading at t=x+3=5 minutes).

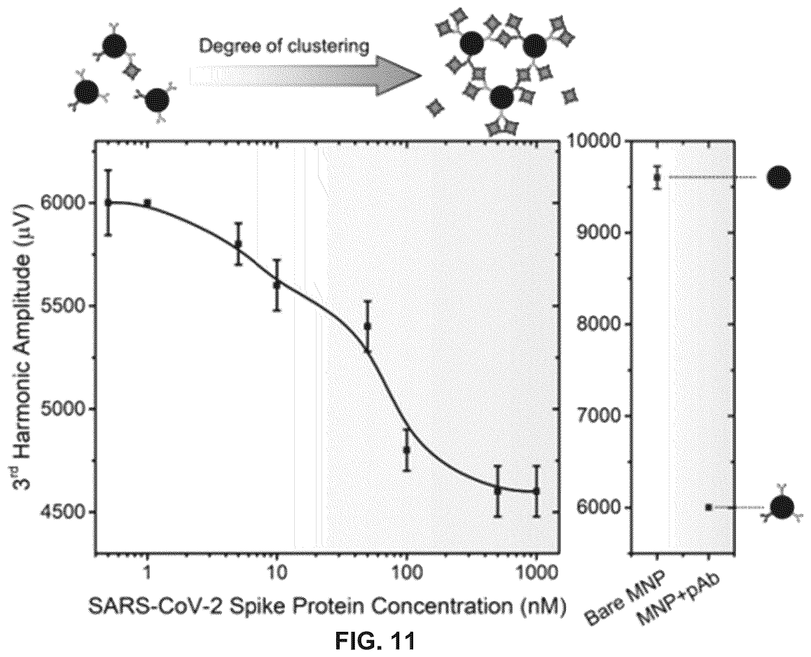

The examples of the fast (e.g., “5-minute”) MPS bioassay strategy/method were also tested on different concentrations of SARS-CoV-2 spike protein, from 1000 nM to 0.5 nM. For example, experiments were conducted on a two-stage lock-in MPS system (FIG. 3B), with an additional voltage gain of around 32 dB for improved bioassay sensitivity. FIG. 11 is a graph illustrating the concentration-response curve of SARS-CoV-2 spike protein, in accordance with one or more techniques of this disclosure.

For example, FIG. 11 illustrates the concentration-response curve of SARS-CoV-2 spike protein tested by a 5-minute MPS bioassay strategy using one or more example techniques described in this disclosure, e.g., according to the method of FIG. 7. In the example shown in FIG. 11, five independent bioassays were carried out at each concentration. Error bars represent standard errors.

In the example shown, the 3^{rd} harmonic amplitude saturates at 500-1000 nM (upper concentration limit) and 0.5-1 nM (lower concentration limit), with a nearly linear response curve between these two limits. As schematically shown in FIG. 11, with higher concentrations of SARS-CoV-2 spike protein added, the degree of MNP clustering increases, and the dynamic magnetic response of MNPs becomes weaker thus, lower harmonic amplitudes are observed.

The averaged 3^{rd} harmonic signals from active experimental samples range from 4500 μV to 6000 μV, for samples with SARS-CoV-2 spike protein concentrations varied from 1000 nM to 0.5 nM. For comparison, the 3^{rd} harmonic amplitudes of bare MNPs (IPG30 without pAb functionalization) and pAb functionalized MNPs (denoted as ‘MNP+pAb’) are 9600 μV and 6000 μV, respectively. Since the pAb conjugated on MNPs impedes the Brownian relaxations, weaker MPS signals are expected from ‘MNP+pAb’ samples. The detection limit of this 5-minute MPS bioassay for SARS-CoV-2 spike protein is somewhere between 1 nM and 5 nM.

Accordingly, this disclosure describes example techniques of the application of higher temperatures (37° C.) and agitation conditions during the MPS bioassay incubation step to accelerate the antibody-antigen specific binding. The thermal energy (heating) and vibrational kinetic energy (agitation) may increase the frequency of successful collisions between SARS-CoV-2 spike pAbs (from MNP surface) and the spike protein molecules, allowing for faster establishment of specific binding equilibrium and shorter diagnosis turnaround time. The example results show that the 5-minute volumetric MPS bioassay strategy described in this disclosure could be an effective way to cut the current COVID-19 diagnosis time from 1 hour to 5 minutes. This quick turnaround in diagnosis may greatly advance surveillance and control strategies for diseases especially for future pandemics. Although this proof-of-concept was demonstrated on the volumetric MPS bioassay platform, it can also be applied to other volumetric biosensors such as nuclear magnetic resonance (NMR) biosensor, ferromagnetic resonance (FMR) biosensor, some types of fluorescent biosensors, gold nanoparticle-based colorimetric assays, or the like.

FIG. 12 is a graph illustrating MPS readings for detection of influenza A virus H1N1, in accordance with one or more techniques of this disclosure. FIG. 12 illustrates a series of plots of the 3^{rd} harmonic amplitudes measured by an MPS system according to the method of FIG. 7 for different concentrations of analytes 820 of influenza A virus H1N1. In the example shown, the higher concentration of target analytes 820 result in lower harmonic signals, e.g., indicating increased clustering of MNPs.

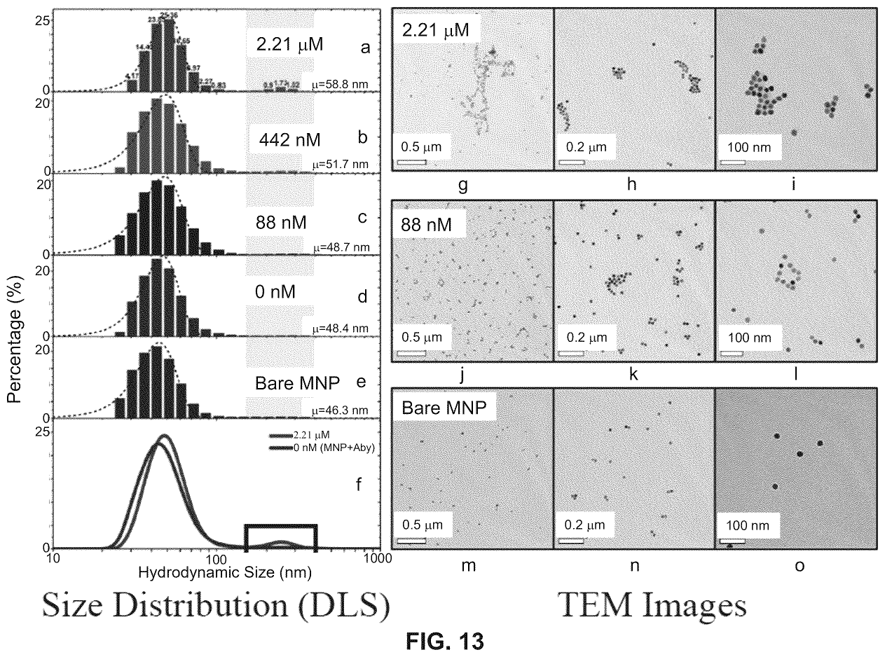

FIG. 13 includes graphs illustrating size distributions (DLS) of MNPs with corresponding TEM (transmission electron microscopy) images corresponding to the MPS measurements of FIG. 12, in accordance with one or more techniques of this disclosure. FIG. 13 illustrates increased hydrodynamic size with higher concentration of analytes 820.

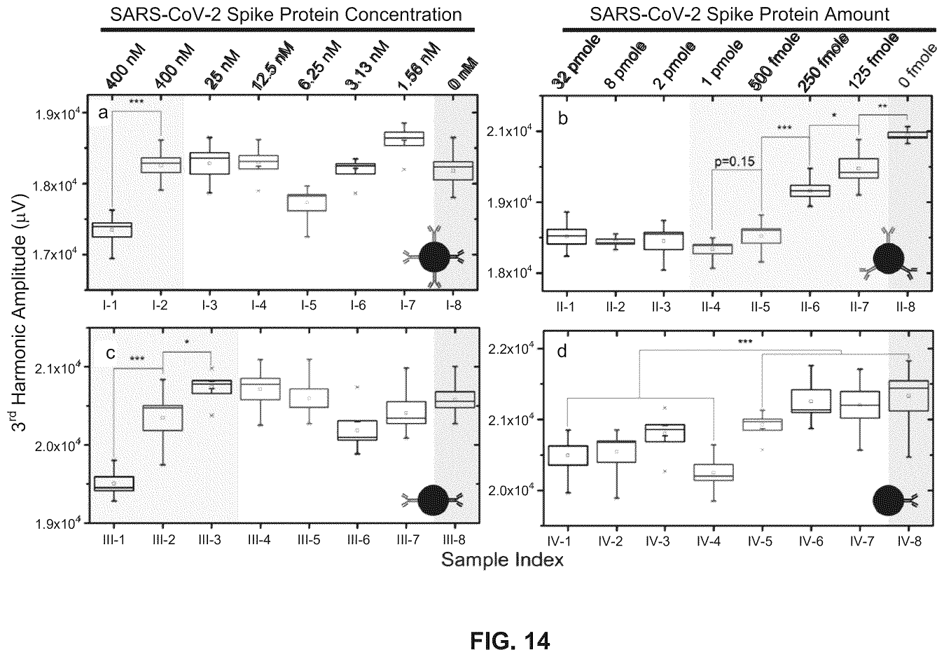

FIG. 14 includes graphs illustrating MPS readings for various SARS-CoV-2 spike protein concentrations and SARS-CoV-2 spike protein amounts, in accordance with one or more techniques of this disclosure. FIG. 14 illustrates graphs (a)-(d) which correspond to functionalizing MNPs 806 with 1, 2, 3, or 4 pAbs per MNP 806, respectively. Graphs (a)-(d) show the mean and range of a plurality of MPS readings for each of the denoted concentrations and amounts of SARS-CoV-2 spike protein. In some examples, functionalizing the MNPs with three pAbs per MNP may provide improved results, e.g., detection sensitivity, and FIG. 14 may illustrate detection of SARS-CoV-2 spike protein with a concentration sensitivity down to at least 1.56 nM (equivalent to 125 fmole) and detection of SARS-CoV-2 nucleocapsid protein with a concentration sensitivity down to at least 12.5 nm (equivalent to 1 pmole).

The techniques described in this disclosure may be implemented, at least in part, in hardware, software, firmware or any combination thereof. For example, various aspects of the described techniques may be implemented within one or more processors, including one or more microprocessors. DSPs, application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

The following examples are described herein.

Example 1: A method including: receiving a sample in a fluid, the fluid comprising a plurality of surface functionalized magnetic nanoparticles (MNPs), wherein the surface functionalized MNPs comprise a probe configured to capture an analyte; increasing a rate of capture by which the probe is configured to capture the analyte within the fluid; and subsequent to increasing the rate of capture, sensing, via volumetric-based magnetic particle spectroscopy (MPS), a magnetic response of the plurality of surface functionalized MNPs, wherein the magnetic response is indicative of whether the sample comprises the analyte.

Example 2: The method of example 1, the method further including: determining that the sample comprises the analyte based on the magnetic response; and generating an output indicating that the sample comprises the analyte based on the determination.

Example 3: The method of example 1, the method further including: determining that the sample does not comprise the analyte based on the magnetic response; and generating an output indicating that the sample does not comprise the analyte based on the determination.

Example 4: The method of any one of any of examples 1-3, wherein the analyte comprises at least one of an antigen, an antibody, a single stranded DNA, and a single stranded RNA, a heavy metal ion, a protease, human coronavirus 229E, human coronavirus OC43, SARS-CoV (2003), HCoV NL63 (2004), HKU1 (2005), MERS-CoV (2102), or SARS-CoV-2.

Example 5: The method of any one of any of examples 1-4, wherein the probe comprises at least one of an antigen, an antibody, a single stranded deoxyribonucleic acid (DNA), a single stranded ribonucleic acid (RNA), or a peptide.

Example 6: The method of any one of examples 1, wherein increasing the rate of capture by which the probe is configured to capture the analyte within the fluid comprises at least one of increasing a temperature of the fluid to an incubation temperature for an incubation time period or agitating the fluid for the incubation time period.

Example 7: The method of example 6, wherein the incubation temperature is in a range of 25° C. to 42° C., 26° C. to 42° C., 27° C. to 42° C., 28° C. to 42° C., 29° C. to 42° C., 30° C. to 42° C., 31° C. to 42° C., 32° C. to 42° C., 33° C. to 42° C., 34° C. to 42° C., 35° C. to 42° C., 36° C. to 42° C., 37° C. to 42° C., 38° C. to 42° C., 39° C. to 42° C., 40° C. to 42° C., or 41° C. to 42° C., or 35° C. to 39° C., or 36° C. to 38° C., or 37° C.

Example 8: The method of example 6 or example 7, wherein the incubation temperature is substantially the same as a physiological temperature.

Example 9: The method of any one of examples 6-8, wherein the incubation time period is in a range of 1 minute to 10 minutes, 1 minute to 9 minutes, 1 minute to 8 minutes, 1 minute to 7 minutes, 1 minute to 6 minutes, 1 minute to 5 minutes, 1 minute to 4 minutes, or 1 minute to 3 minutes.

Example 10: The method of any one of examples 6-9, wherein the incubation time period is substantially equal to 3 minutes.

Example 11: The method of any one of examples 6-10, wherein agitating the fluid comprises at least one of shaking the fluid, stirring the fluid, or applying a rotating magnetic field to the fluid.

Example 12: A bioassay system including: a volumetric-based magnetic particle spectroscopy (MPS) device configured to determine a magnetic response indicative of whether a sample comprises an analyte; a fluid comprising a plurality of surface-functionalized magnetic nanoparticles (MNPs), wherein the fluid is configured to receive the sample, wherein the surface functionalized MNPs comprise a probe configured to capture the analyte; and an incubator configured to increase a rate of capture by which the probe is configured to capture the analyte within the fluid subsequent to the fluid receiving the sample, wherein the volumetric-based MPS device is configured to determine the magnetic response subsequent to increasing the rate of capture.

Example 13: The bioassay system of example 12, wherein the volumetric-based MPS device is further configured to: determine whether the sample comprises the analyte based on the magnetic response; and generate an output indicating whether the fluid comprises the analyte based on the determination.

Example 14: The bioassay system example 12 or example 13, wherein the analyte comprises at least one of an antigen, an antibody, a single stranded DNA, and a single stranded RNA, a heavy metal ion, a protease, human coronavirus 229E, human coronavirus OC43, SARS-CoV (2003), HCoV NL63 (2004), HKU1 (2005), MERS-CoV (2102), or SARS-CoV-2.

Example 15: The bioassay system of any one of examples 12-14, wherein the probe comprises at least one of an antigen, an antibody, a single stranded deoxyribonucleic acid (DNA), a single stranded ribonucleic acid (RNA), or a peptide.

Example 16: The bioassay system of any one of examples 12-15, wherein the incubator is configured to at least one of increase a temperature of the fluid to an incubation temperature for an incubation time period or to agitate the fluid for the incubation time period.

Example 17: The bioassay system of example 16, wherein the incubation temperature is in a range of 25° C. to 42° C., 26° C. to 42° C., 27° C. to 42° C., 28° C. to 42° C., 29° C. to 42° C., 30° C. to 42° C. 31° C. to 42° C. 32° C. to 42° C. 33° C. to 42° C. 34° C. to 42° C. 35° C. to 42° C. 36° C. to 42° C. 37° C. to 42° C. 38° C. to 42° C. 39° C. to 42° C. 40° C. to 42° C. or 41° C. to 42° C., or 35° C. to 39° C., or 36° C. to 38° C., or 37° C.

Example 18: The bioassay system of example 16 or example 17, wherein the incubation temperature is substantially the same as a physiological temperature.

Example 19: The bioassay system of any one of examples 16-18, wherein the incubation time period is in a range of 1 minute to 10 minutes, 1 minute to 9 minutes, 1 minute to 8 minutes, 1 minute to 7 minutes, 1 minute to 6 minutes, 1 minute to 5 minutes, 1 minute to 4 minutes, or 1 minute to 3 minutes.

Example 20: The bioassay system of any one of examples 16-19, wherein the incubation time period is substantially equal to 3 minutes.

Example 21: The bioassay system of any one of examples 16-20, wherein the incubator is configured to at least one of shake the fluid, stir the fluid, or apply a rotating magnetic field to the fluid.

Example 22: A volumetric-based magnetic particle spectroscopy (MPS) device including: at least one conductive excitation coil, the at least one conductive excitation coil configured to generate an alternating magnetic field including a plurality of frequencies; a sample mount configured to position a fluid within the at least one conductive excitation coil, the fluid comprising a plurality of surface-functionalized magnetic nanoparticles (MNPs), wherein the fluid is configured to receive a sample, wherein the surface functionalized MNPs comprise a probe configured to capture an analyte; an incubator configured to increase a rate of capture by which the probe is configured to capture the analyte; at least one sensing conductive coil configured to determine a magnetic response of the fluid positioned within the sample mount to the alternating magnetic field subsequent to increasing the rate of capture; processing circuitry configured to determine whether the sample comprises the analyte based on the magnetic response.

Example 23: The volumetric-based MPS device of example 22, wherein the volumetric-based MPS device is configured to determine the magnetic response of the fluid positioned within the sample mount to the alternating magnetic field in less than five minutes from the sample being added to the fluid.

Various examples of the invention have been described. These and other examples are within the scope of the following claims.

1. A method comprising:
receiving a sample in a fluid, the fluid comprising a plurality of surface functionalized magnetic nanoparticles (MNPs), wherein the surface functionalized MNPs comprise a probe configured to capture an analyte;
increasing a rate of capture by which the probe is configured to capture the analyte within the fluid; and
subsequent to increasing the rate of capture, sensing, via volumetric-based magnetic particle spectroscopy (MPS), a magnetic response of the plurality of surface functionalized MNPs, wherein the magnetic response is indicative of whether the sample comprises the analyte.

2. The method of claim 1, the method further comprising:
determining that the sample comprises the analyte based on the magnetic response; and
generating an output indicating that the sample comprises the analyte based on the determination.

3. The method of claim 1, the method further comprising:
determining that the sample does not comprise the analyte based on the magnetic response; and
generating an output indicating that the sample does not comprise the analyte based on the determination.

4. The method of claim 1, wherein the analyte comprises at least one of an antigen, an antibody, a single stranded DNA, and a single stranded RNA, a heavy metal ion, a protease, human coronavirus 229E, human coronavirus OC43, SARS-CoV (2003), HCoV NL63 (2004), HKU1 (2005), MERS-CoV (2102), or SARS-CoV-2.

5. The method of claim 1, wherein the probe comprises at least one of an antigen, an antibody, a single stranded deoxyribonucleic acid (DNA), a single stranded ribonucleic acid (RNA), or a peptide.

6. The method of claim 1, wherein increasing the rate of capture by which the probe is configured to capture the analyte within the fluid comprises at least one of increasing a temperature of the fluid to an incubation temperature for an incubation time period or agitating the fluid for the incubation time period.

7. The method of claim 6, wherein the incubation temperature is in a range of 25° C. to 42° C., 26° C. to 42° C., 27° C. to 42° C., 28° C. to 42° C., 29° C. to 42° C., 30° C. to 42° C., 31° C. to 42° C., 32° C. to 42° C., 33° C. to 42° C., 34° C. to 42° C., 35° C. to 42° C., 36° C. to 42° C., 37° C. to 42° C., 38° C. to 42° C., 39° C. to 42° C., 40° C. to 42° C., or 41° C. to 42° C., or 35° C. to 39° C., or 36° C. to 38° C., or 37° C.

8. The method of claim 6, wherein the incubation temperature is substantially the same as a physiological temperature.

9. The method of claim 6, wherein the incubation time period is in a range of 1 minute to 10 minutes, 1 minute to 9 minutes, 1 minute to 8 minutes, 1 minute to 7 minutes, 1 minute to 6 minutes, 1 minute to 5 minutes, 1 minute to 4 minutes, or 1 minute to 3 minutes.

10. The method of claim 6, wherein the incubation time period is substantially equal to 3 minutes.

11. The method of claim 6, wherein agitating the fluid comprises at least one of shaking the fluid, stirring the fluid, or applying a rotating magnetic field to the fluid.

12. A bioassay system comprising:
a volumetric-based magnetic particle spectroscopy (MPS) device configured to determine a magnetic response indicative of whether a sample comprises an analyte;
a fluid comprising a plurality of surface-functionalized magnetic nanoparticles (MNPs), wherein the fluid is configured to receive the sample, wherein the surface functionalized MNPs comprise a probe configured to capture the analyte; and
an incubator configured to increase a rate of capture by which the probe is configured to capture the analyte within the fluid subsequent to the fluid receiving the sample,
wherein the volumetric-based MPS device is configured to determine the magnetic response subsequent to increasing the rate of capture.

13. The bioassay system of claim 12, wherein the volumetric-based MPS device is further configured to:
determine whether the sample comprises the analyte based on the magnetic response; and
generate an output indicating whether the fluid comprises the analyte based on the determination.

14. The bioassay system of claim 12, wherein the analyte comprises at least one of an antigen, an antibody, a single stranded DNA, and a single stranded RNA, a heavy metal ion, a protease, human coronavirus 229E, human coronavirus OC43, SARS-CoV (2003), HCoV NL63 (2004), HKU1 (2005), MERS-CoV (2102), or SARS-CoV-2.

15. The bioassay system of claim 12, wherein the probe comprises at least one of an antigen, an antibody, a single stranded deoxyribonucleic acid (DNA), a single stranded ribonucleic acid (RNA), or a peptide.

16. The bioassay system of claim 12, wherein the incubator is configured to at least one of increase a temperature of the fluid to an incubation temperature for an incubation time period or to agitate the fluid for the incubation time period.

17. The bioassay system of claim 16, wherein the incubation temperature is in a range of 25° C. to 42° C., 26° C. to 42° C., 27° C. to 42° C., 28° C. to 42° C., 29° C. to 42° C., 30° C. to 42° C., 31° C. to 42° C., 32° C. to 42° C., 33° C. to 42° C., 34° C. to 42° C., 35° C. to 42° C., 36° C. to 42° C., 37° C. to 42° C., 38° C. to 42° C., 39° C. to 42° C., 40° C. to 42° C., or 41° C. to 42° C., or 35° C. to 39° C., or 36° C. to 38° C., or 37° C.

18. The bioassay system of claim 16, wherein the incubation temperature is substantially the same as a physiological temperature.

19. The bioassay system of claim 16, wherein the incubation time period is in a range of 1 minute to 10 minutes, 1 minute to 9 minutes, 1 minute to 8 minutes, 1 minute to 7 minutes, 1 minute to 6 minutes, 1 minute to 5 minutes, 1 minute to 4 minutes, or 1 minute to 3 minutes.

20. The bioassay system of claim 16, wherein the incubation time period is substantially equal to 3 minutes.

21. The bioassay system of claim 16, wherein the incubator is configured to at least one of shake the fluid, stir the fluid, or apply a rotating magnetic field to the fluid.

22. A volumetric-based magnetic particle spectroscopy (MPS) device comprising:
at least one conductive excitation coil, the at least one conductive excitation coil configured to generate an alternating magnetic field including a plurality of frequencies;
a sample mount configured to position a fluid within the at least one conductive excitation coil, the fluid comprising a plurality of surface-functionalized magnetic nanoparticles (MNPs), wherein the fluid is configured to receive a sample, wherein the surface functionalized MNPs comprise a probe configured to capture an analyte;
an incubator configured to increase a rate of capture by which the probe is configured to capture the analyte;
at least one sensing conductive coil configured to determine a magnetic response of the fluid positioned within the sample mount to the alternating magnetic field subsequent to increasing the rate of capture;
processing circuitry configured to determine whether the sample comprises the analyte based on the magnetic response.

23. The volumetric-based MPS device of claim 22, wherein the volumetric-based MPS device is configured to determine the magnetic response of the fluid positioned within the sample mount to the alternating magnetic field in less than five minutes from the sample being added to the fluid.
